(19) United States
(12) Patent Application Publication
(10) Pub. No.: US 2013/0225430 A1
(43) Pub. Date: Aug. 29, 2013

(54) METHODS AND COMPOSITIONS FOR TREATMENT OF TOX-3 AND TFF-1 MEDIATED CANCER PATHOGENESIS

(71) Applicant: CEDARS-SINAI MEDICAL CENTER, (US)

(72) Inventors: Jonathan Kaye, Los Angeles, CA (US); Akop Seksenyan, Los Angeles, CA (US)

(73) Assignee: CEDARS-SINAI MEDICAL CENTER, Los Angeles, CA (US)

(21) Appl. No.: 13/761,029

(22) Filed: Feb. 6, 2013

Related U.S. Application Data
(60) Provisional application No. 61/596,069, filed on Feb. 7, 2012.

(51) Int. Cl. C12Q 1/68; G01N 33/68

(52) U.S. Cl. CPC C12Q 1/6886; G01N 33/6872; G01N 33/6893
USPC 506/9; 435/6.12; 435/7.1; 435/7.23

(57) ABSTRACT

The present invention relates to biomarkers useful for the diagnosis, prognosis and treatment of cancer. In different embodiments, the invention relates to TOX3 and related biomarkers, such as TFF1, TFF3, AGR2, SCUBE2, CEACAM6, TSPAN1 or CXCR4. TOX3 and these related biomarkers are also useful for the characterization of different breast cancer disease subtypes. Also described herein are inventive compositions and methods drawn to the use of anti-TOX3 antibodies, inducible TOX3 transgenic animal models, TOX3 nucleic acids, peptides, and small molecules for detection and modulation of TOX3 function and/or expression.

### BACKGROUND

Breast cancer remains a serious public health problem. Aside from skin cancer, breast cancer is the most common form of cancer in women, with a lifetime incidence rate in the US population of approximately 13%. Breast cancer also remains one of the top ten causes of death for women in the US, and the second leading cause of cancer deaths in this population. Like all forms of cancer, breast cancer can be considered as a molecular reprogramming of the normal cell. Thus, understanding the gene regulatory networks that exist in breast cancer cells is of fundamental importance for establishing new diagnostic and therapeutic approaches.

Mutations in BRCA1 or BRCA2 genes impart a very high risk for development of breast cancer. However, such mutations exist in the population at low frequency (and generally act as recessive cancer genes), and thus cannot account for the majority of breast cancers. Mutations in other genes, including PT53, PTEN, STK11, CDH1, also impart significantly increased risk of disease. However, even together with BRCA1 and BRCA2, these mutations may only account for 20% of familial disease (1). Thus, multiple additional genetic factors account for the observed disease incidence. In addition, the complexity of disease means that there can be additive and synergistic effects of changes in other mediators, even in the context of BRCA1 and BRCA2 mutations as discussed herein.

Using microarray analysis, the inventors have identified early changes in gene expression that take place in precursor thymocytes as they traversed a developmental checkpoint-termed positive selection. These studies led to identification of a gene encoding a nuclear protein subsequently designated TOX (Thymocyte selection-associated HMG-box protein) (2). The inventors characterized mice deficient in TOX and showed that this nuclear factor is required for development of a number of key aspects of the immune system including development of CD4 T cells, lymph nodes, and NK cells (9). Together the data indicate that TOX is a key regulator of precursor cell differentiation in various contexts, presumably by regulating gene expression.

This role for TOX genes in cellular differentiation implicates a role for TOX3 in the branching cellular morphogenesis and fat pad invasion—key development and regulatory processes for formation and maintenance breast organ structures and tissue. In this context, TOX3 may serve as an important development factor guiding these processes. Perturbations in TOX3 expression may contribute to cancer initiation, progression, or defining the pathology of cancer molecular subtypes.

As described herein, TOX3 has a specific role in the development of ER⁺ luminal epithelial cells of the ductal chambers within the breast. Further results indicate that TOX3 induces expression of cancer-related genes involved in cellular migration, such as trefoil factor 1 (TFF1) and chemokine receptor CXCR4. Interestingly, TOX3 mediated induction of these genes appears to function independent of estrogen E2 (also known as 17β-estradiol and oestradiol) activation of estrogen receptor (ER). This is shown by tamoxifen resistance and fulvestant sensitivity for TOX3 mediated TFF1 induction, and ChIP studies where binding of ER at various estrogen response element (ERE) sites is observed in TOX3 expressing cells under estrogen depletion conditions. Together, these results are strong evidence of an important role for TOX3 in breast cancer pathology, including a hereto undiscovered mechanism for ER activation and binding.

### BRIEF DESCRIPTION OF THE DRAWINGS

FIG. 1. Depiction of divergent roles of TOX family members in various tissues of the body.

FIG. 2. Microarray of TOX3 expression. (A): Data analyzing 51 breast cancer cell lines (B): data analyzing 118 aggressively-treated early stage breast tumors. Both sets of data are shown as heat maps, ordered based on expression of the TOX3 gene, with high to low expression shown bottom to top.

FIG. 3. Graphical representation of TOX and TOX3 expression in different cell lines. Quantitative RT-PCR was used for specific detection of TOX and TOX3 in human cell lines. (A): Expression of TOX in (L to R) breast cancer cell lines, ZR-75-1, SKBr3, MDA-MB231 and T-Cell line, MOLT4. (B): Expression of TOX3, in those same breast cancer or T-cell lines, as indicated. For all experiments, relative gene expression is normalized to expression of the MRPL19 housekeeping gene.

FIG. 4. Graphical representation TOX3 expression in various cell lines and tissue samples. (A): Additional quantitative RT-PCR experiments measuring TOX3 gene expression in various tissues and cell lines. Normal breast tissue samples are in blue, breast cancer cell line ZR75-1 in yellow, and different tumor samples in red. The sample derived from a non-cancer patient was arbitrarily set to 1, with relative expression across samples normalized against this sample. (B): Depicts the same data with a reduced scale on the y-axis.

FIG. 5. Identification and characterization of TOX3 splice variants. (A): The predicted amino acid sequences of the N-terminus encoded by variant forms of TOX3 mRNA. Gray indicates identity (variants are identical throughout subsequent C-terminal sequence, not shown). (B): PCR gel of variant forms of TOX3 from breast cancer cell lines ZR75-1 and BC.MFT. Specific primer designs for each variant allow detection a amplification of a 170 base pair fragment unique to variant 1, and amplification of a 186 base pair fragment for variant 2. (C): Western blot detection using novel rabbit monoclonal anti-TOX3 antibody AJ33 specifically identifies TOX3 in HEK-293 or MCF-7 cell lines transfected with vector control or TOX3 vector in as indicated.

FIG. 6. Staining for TOX3 in breast tissue microarray. (A): Various depictions of normal, non-tumor samples, wherein anti-TOX3 AJ33 antibody staining does not show TOX3 staining. (B): In various tumor samples, AJ33 detection of TOX3 results in dark compact intracellular positive staining.

FIG. 7. Staining for TOX3 in breast tumor tissue samples. (A): Tissue staining of tumor samples using AJ33 demonstrates high levels of TOX3 expression in tumor, as shown by positive staining. (B): By contrast, adjacent normal breast tissue shows almost no positive staining for TOX using AJ33 antibody. However, some cells within adjacent normal breast tissue do exhibit light positive staining for TOX3, as indicated by the red arrows.

FIG. 8. Staining for TOX3 in breast tumor tissue samples. (A): Higher magnification of AJ33 positive staining of TOX3 in tumor. (B): Highest magnification of AJ33 positive staining of TOX3 in tumor. Inset panel shows fluorescent microscopy. (C): Adjacent normal breast tissue demonstrates virtually no positive staining for TOX3 using AJ33 antibody. However, occasional positive staining in individual cells, as shown by red arrows.

FIG. 9. Cell sorting identifies subsets of murine mammary epithelial cells in virgin females. (A): Flow cytometry cell sorting of Lin⁻CD24⁺ cells using antigen markers, CD61 and CD29, helps identify three distinct populations of undifferentiated cells in mammary epithelium. This includes precursors for group I—luminal cells (CD61^{hi}CD29^{med}); group II—‘stem’ cells and basal layer cells (CD61^{med}CD29^{hi}); and group III—mature luminal cells) (CD61^{lo}CD29^{lo}), as indicated. (B): Measurement of TOX3 expression using qRT-PCR among groups I, II, and II, demonstrate markedly higher expression of TOX3 in group III, mature luminal cells.

FIG. 10. Further sorting of luminal populations demonstrates distinct populations of specific marker expression. (A): Sorting among Lin⁻CD24^{hi} luminal populations according to antigen markers SCA-1 and CD24 demonstrate high levels of variable SCA-1 expression, described as SCA-1^{hi} (9%) and SCA-1^{low} (69%). (B): Further sorting along antigen markers CD61 and CD29 demonstrates a predominantly CD61^{lo}CD29^{lo} antigenic profile among SCA-1^{hi} expressing cells (73% of SCA-1^{hi} cells, 6.6% of total Lin⁻CD24^{hi}SCA-1^{hi} luminal population). (C): By contrast, SCA-1^{lo} expressing cells contain a much smaller population of cells with a CD61^{lo}CD29^{lo} antigenic profile (21%, 14.5% of total Lin⁻CD24^{hi}SCA-1^{low} luminal population).

FIG. 11. Gene expression is enriched in the ER⁺subset of ER⁺luminal cells in normal mammary epithelium. Quantitative real-time PCR experiments further demonstrate that SCA-1^{hi} expressing cells display a remarkably different expression profile compared to SCA-1^{lo} ER⁺ luminal cells. Estrogen receptor α (ESR), transcription factor FOXA1, and TOX3 were all enriched by at least 7-fold in SCA-1^{hi} ER⁺ cells compared to SCA-1^{lo} ER⁺ cells, as indicated. For all experiments, SCA-1^{hi} expression levels were normalized against SCA-1^{lo} expression measurements.

FIG. 12. TOX3 can influence mammary epithelial cell development in Cre-activated TOX3 transgenic mice. (A): TOX3 cassette was inserted into inducible Cre-loxₚ IRES vector designed as shown. Expression cassette is flanked by Sfil restriction sites, and contains dual reporters, RFP and Luciferase-eGFP. (B): Transgenic mice engineered to include the TOX3 victor are viable and exhibit normal morphology, including fluorescence, as shown. (C): Transgenic mice (Tg⁺) display red RFP fluorescence both in the absence or presence of Cre (Tg⁺Cre⁺), while wild-type (Tg⁻) mice do not. (D): Specific expression of TOX3 is induced by Cre in transgenic animals (Tg⁺Cre⁺), but is not expressed in the absence of Cre (Tg⁺) or wild-type (Tg⁻) animals.

FIG. 13. Principal component analysis indicates that molecular heterogeneity of BC may relate to cell of origin and/or differentiation state of tumors. (A): Breast cancer samples organized according to molecular sub-types, classified according to gene expression profiles using principal component analysis (PCA). (B): Cellular contingents of the normal mammary gland demonstrates various cell types present in mammary gland tissue and organized using PCA (from Guedj, et al., Oncogene. 2011 (ePub)).

FIG. 14. Principal component analysis on publicly available breast tumor data. (A): Application of principal component analysis to microarray data for 1391 breast tumor samples demonstrates that the vast majority of 957 may be classified into subtypes of “core” tumors, 374 “outlier” tumors, and 60 “mixed” tumors, as indicated for different subtypes, molecular-apocrine (mApo), basal-like (basL), normal-like (normL), luminal A, B, and C (lumA, lumB, and lumC, respectively). (B): Input dataset used for principal component analysis.

FIG. 15. Expression of TOX3 in breast cancer molecular subtypes. (A): Relative expression demonstrate higher of expression of TOX3 in several subtypes, such as lumA, lumB, lumC, and mApo, compared to normL, and basL, as shown. Data is relative expression, log-transformed and represents the average of 3 microarray probes. (B): Same data as sub-figure A, presented in pie-chart form to demonstrate distribution across sub-types. (C): Narrowing the distribution of sub-figure B to focus only on subtypes with upper 15% of TOX3 expression, enriches for luminal and mApo subtypes, with dramatically lower depletion in basL and normL subtypes.

FIG. 16. Survivorship distribution of patients harboring different cancer molecular subtypes. Each panel depicts time to death for TOX3^{hi} expression (>0.8 quantile), along with TOX3^{lo} expression (≦0.8 quantile). Measurements reached statistical significance for lumB (p=0.01) and mApo (p=0.068) subtypes, with P values set as null hypothesis of similar survivorship.

FIG. 17. TOX3 in patient sample tissue array. (A): Estrogen receptor (ER) and herceptin (Her2) protein expression distribution in tissue microarray, distribution shown as pie chart. Tissue array contains 188 patient samples, with the vast majority displaying invasive ductal carcinoma. (B): Distribution of TOX3^{hi} expression across sample types. TOX3^{hi} (>10%++ staining) was identified ˜16% of tumors. Individual percentages for TOX3^{hi} in ER and Her2 categories are indicated (25% ER⁺Her2⁺, 11% ER⁺Her2⁻, 15% ER⁻Her2⁺, and 8% ER⁻Her2⁻), along six molecular subtypes that can be associated with particular ER and Her2 antigenic profiles (molecular-apocrine (mApo), basal-like (basL), normal-like (normL), luminal A, B, and C (lumA, lumB, and lumC, respectively)).

FIG. 18. General study design for TOX3 gene induction studies. Breast cancer cell line ER⁺MCF-7 were transfected with TOX3 or vector control, then placed in estrogen depleted media 8 hours post-transfection, with sorting of GFP⁺ cells 36 hours post-transfection. Over 90 genes were found to be upregulated by at least 3-fold, including those as indicated.

FIG. 19. Measurement of TFF1 induction by TOX3. Breast cancer cell line, MCF-7 was transfected with TOX3 or vector control in estrogen depleted media, and TFF1 expression was measured via qRT-PCR. Normalized against vector control TFF1 expression, TOX3 vector induced TFF1 at nearly equivalent levels as 10 nM estrogen stimulated vector control (˜10× fold increase). Combined TOX3 transfection with 10 nM estrogen stimulation was nearly 20× higher than unstimulated vector control.

FIG. 20. TOX3-mediated induction of TFF1 in stable MCF-7 cell transfectants that express TOX3. TOX3 induction of TFF1 was recapitulated in 2 stable transfectant cell lines (T3-1 and T3-2), as shown by the increase in relative expression levels compared to vector control (V). Unstimulated T3-1 and T3-2 cells lines expressed TFF1 at nearly the same levels as vector control stimulated by estrogen. In these experiments, cell lines were placed in estrogen depleted media for 48 h, and where applicable, stimulated with estrogen E2 for 24 hours.

FIG. 21. TOX3 induction of TFF1 is tamoxifen sensitive, but fulvestrant resistant. (A) Under normal culture conditions, both tamoxifen and fulvestrant cause a decrease in TOX-3 mediated induction of TFF1 expression compared to untreated control cells. Similarly, estrogen depletion causes a decrease in TFF1 expression, including untreated cells, and those treated with tamoxifen and fulvestrant. In TOX3 transfected cells, a significant increase in TFF1 expression occurs in both untreated and tamoxifen treated cells. However, this increase does not occur in cells treated with fulvestrant, thereby demonstrating that TOX3 induction of TFF1 is tamoxifen resistant, but fulvestrant sensitive. (B) Another representation of experiment showing TOX3 induction of TFF1 is tamoxifen sensitive, but fulvestrant resistant. MCF7 cells were transfected with a TOX3 expression vector or vector control, switched to estrogen depleted-medium after 8 hours in the presence or absence of the indicated drug, and analyzed by quantitative RT-PCR for TFF1 gene expression after an additional 36 hours in culture. As a control, the effects of these drugs on cells in normal medium (i.e. without specific removal of estrogen or phenol red) are shown (blue, not depleted).

FIG. 22. Estrogen receptor sequence. Depiction of estrogen receptor a (ERa) protein sequence, including sub-domains and phosphorylation sites. Phosphorylation of estrogen receptor occurs at several sites (designated “P” as shown), including at serine 118 (s118). Specific detection of ERa phosphorylation was measured via Western blot, and observed under normal growth conditions and in estrogen depleted media for both TOX3 and vector transfected cells, as shown.

FIG. 23. Promoter sequence of TFF1. (A): TFF1 promoter sequence contains three estrogen receptor elements (ERE I, ERE II, and ERE III). ERE II and III are located several kilobases upstream of the TFF 1 transcription start site. Each ERE site is highly conserved among different animal species as shown. (B): Existing studies have characterized the binding of ERα to ERE I (designated as “PR”), ERE II, and ERE III via chromatin immunoprecipitation of ER (ER-ChIP) along various lengths of the TFF1 and TMPRSS3 flanking regions as indicated. Residual binding is observed even after tamoxifen and fulvestrant treatment (from Welboren et al. The EMBO Journal (2009) 28, 1418-1428) FIG. 24. ChIP-qPCR in MCF-7 breast cancer cells at TFF1 locus. Studies by the inventors demonstrate that TOX3 transfection is capable of promoting ERa binding to the ERE I (designated as “Promoter”), ERE II and ERE III sites, similarly to E2, compared to vector control.

FIG. 25. TOX3 induces CXCR4 in MCF-7 cells. Cell sorting for CXCR4 and GFP demonstrate that TOX3 induces expression of CXCR4 chemokine receptor. Whereas only 3% of vector-GFP transfected cells were CXCR4⁺, 40% of TOX3-GFP transfected cells were CXCR4⁺.

FIG. 26. TOX3 transfection of MCF7 cells promotes cell migration in response to serum. Using a transwell assay system, cells transfected with TOX3 migrate through matrigel in response to fetal calf serum (designated as “FCS”), compared to unstimulated TOX3 or vector transfected cells and stimulated vector transfected cells.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), and Remington: The Science and Practice of Pharmacy (Gennaro ed. 20th edition, Williams & Wilkins PA, USA) (2000) provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Antibody” or “antibodies,” as used herein, refer to immunoglobin proteins that bind specifically to a protein, without significant cross-reaction with other proteins. “Antibodies” (Abs) is used in the broadest sense and specifically covers, without limitation, intact monoclonal antibodies, polyclonal antibodies, multispecific antibodies (e.g. bispecific antibodies) formed from at least two intact antibodies, and antibody fragments so long as they exhibit the desired biological activity.

“Biomarker,” as used herein, refers to a molecular indicator that is associated with a particular pathological or physiological state. As one example, a “biomarker” can be used as a molecular indicator for cancer. In one example, the biomarker is an indicator for molecular subtypes of breast cancer that can be linked to TOX3 expression. Examples of “biomarkers” include, but or not limited to including a protein, polynucleotide, allele, or transcript of TOX3, TFF1, TFF3, AGR2, SCUBE2, CEACAM6, TSPAN1 or CXCR4. A “biomarker” of the present invention may be detected in a tumor, tissue, or cell sample obtained from a subject with a disease and/or condition, such as cancer, or from a subject suspected of having a disease and/or condition.

“Cancer” and “cancerous,” as used herein, describes the physiological condition in mammals that is typically characterized by unregulated cell growth. Examples of cancer include, but are not limited to breast, osteosarcoma, cancer, colon cancer, lung cancer, prostate cancer, hepatocellular cancer, gastric cancer, pancreatic cancer, cervical cancer, ovarian cancer, liver cancer, and bladder cancer, cancer of the urinary tract, thyroid cancer, renal cancer, carcinoma, melanoma, head and neck cancer, brain cancer, or any TOX3 related cancer.

“Gene delivery vector,” as used herein, refers generally to a nucleic acid construct that is capable of directing the expression particular gene or genetic construct. One example is an antisense molecule cognate to a target sequence of interest, as described in, for example, Molecular Biotechnology Principles and Applications of Recombinant DNA, Ch. 21, pp. 555-590 (ed. B. P. Glick and J. J. Pasternak, 2nd ed. 1998); Jolly, Cancer Gene Ther. 1:51-64 (1994); Kimura, Human Gene Ther. 5:845-852 (1994); Connelly, Human Gene Ther. 6:185-193 (1995); and Kaplitt, Nat. Gen. 6:148-153 (1994).

“Instructions for use,” as used herein, typically include a tangible expression describing the technique to be employed in using the components of the kit to effect a desired outcome, such as to treat ischemia. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

“Nucleic acid,” as used herein, means a polynucleotide such as a single- or double-stranded DNA or RNA molecule including, for example, genomic DNA, cDNA, and mRNA. The term nucleic acid encompasses nucleic acid molecules of both natural and synthetic origin as well as molecules of linear, circular, or branched configuration representing either the sense or antisense strand, or both, of a native nucleic acid molecule.

“Overexpression,” as used herein refers to expression of a gene and/or its encoded protein in a test cell, such as a tumor cell, that is higher than the value measured from a reference cell, such as a non-tumor cell. For example, a tumor cell that “overexpresses” a protein is one that has significantly higher levels of that protein compared to a normal cell of the same tissue type.

“Polypeptide” or “peptide,” as used herein refers comprising at least a part (i.e., the whole or a part) of the amino acid sequence.

“Package,” as used herein, refers to a suitable solid matrix or material such as glass, plastic, paper, foil, and the like, capable of holding the individual kit components. Thus, for example, a package can be a glass vial used to contain suitable quantities of an inventive composition containing a polyphenol analog.

“Packaging material,” as used herein, refers to one or more physical structures used to house the contents of the kit, such as inventive compositions and the like. The packaging material is constructed by well-known methods, preferably to provide a sterile, contaminant-free environment.

“Therapeutic agents,” as used herein, refer to agents that prevent initiation, reduce likelihood, slow progression, or eliminate the presence of a disease and/or condition in a subject. Examples of therapeutic agents or modulators include, but are not limited to small interfering RNA, antisense molecules, antibodies or antibody fragments, proteins or polypeptides as well as small molecules.

“Therapeutically effective amount” as used herein, refers to that amount which is capable of achieving beneficial results in a patient with cancer. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein, refer to both therapeutic treatment and prophylactic or preventative measures, wherein the object is to prevent, slow down and/or lessen the disease even if the treatment is ultimately unsuccessful. Those in need of treatment include those already with cancer as well as those prone to have cancer or those in whom cancer is to be prevented. For example, in cancer treatment, a therapeutic agent or modulator may directly decrease the pathology of cancer cells, or render the tumor cells more susceptible to treatment by other therapeutic agents or by the subject's own immune system.

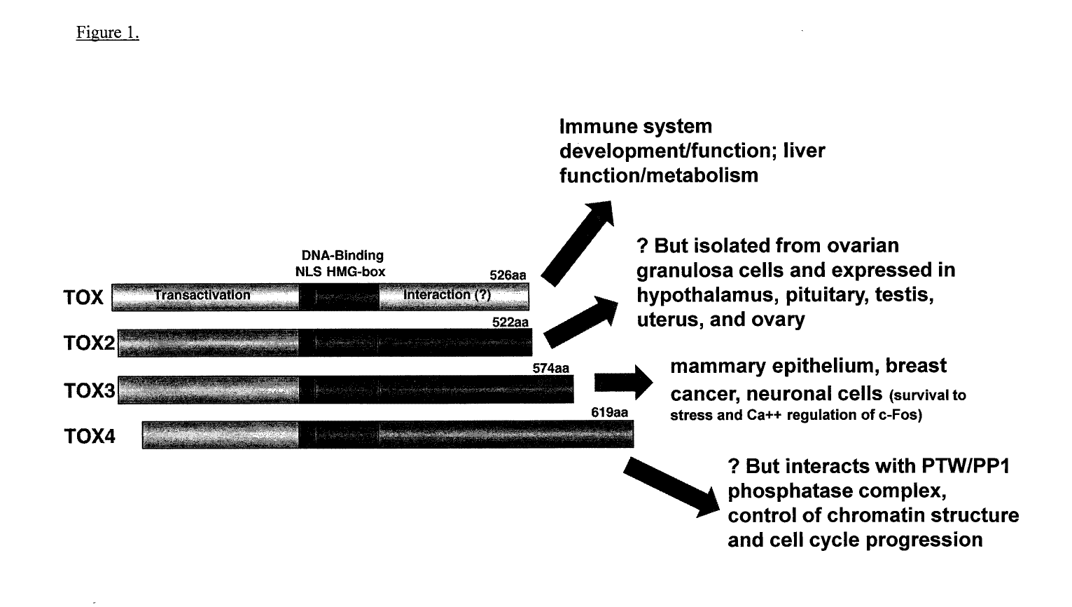

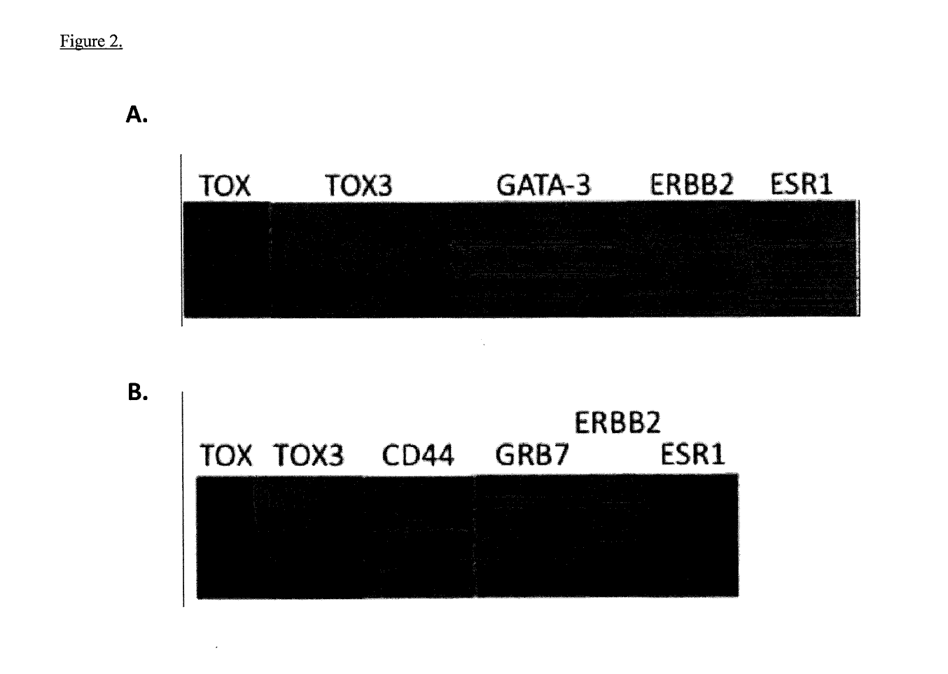

TOX3 is a member of the TOX family and is a sub-group of the HMG-box of proteins, which are involved in binding to chromatin and altering transcription. The TOX family members includes TOX1-4 (5), ranging in lengths from 526aa to 619 aa, and which possess divergent roles in various tissues (FIG. 1). These proteins contains a single centrally-located DNA binding motif known as an HMG-box, named after the motif found in canonical HMGB proteins. The HMG-box now defines a superfamily of proteins (which have 47 family members located in the human genome). Despite having diverse functions, these proteins share some general characteristics of DNA binding. HMG-box domains, including TOX, fold into three α-helices that form a concave L-shaped structure that binds the minor groove of DNA. HMG-box proteins also bind distorted DNA structures and often can induce bending and unwinding of the DNA helix to fit the protein domain structure.

Two general classes of HMG-box proteins have been identified based on their mode of binding to DNA: (a) those that exhibit sequence-specific binding and (b) those that bind DNA in a sequence-independent but structure-dependent fashion. The latter class of proteins include the canonical HMGB proteins themselves, while the former include transcriptional regulators, such as LEF-1. Both kinds of proteins, however, play roles in regulating gene expression, often by inducing or stabilizing architectural changes in chromatin and facilitating nucleoprotein complex formation. HMG-box proteins may also augment other nuclear functions that benefit from architectural changes in DNA, including antigen receptor gene rearrangement (3) and chromatin remodeling (4). By inspection of key residues in the HMG-box domain (TOX-box), TOX is almost certainly a member of the sequence-independent DNA-binding family (5). As a structure dependent HMG-box protein, TOX can bind distorted (cisplatinated) or bent (circular) dsDNA However, lack of an internal Met wedge makes TOX a poor bender of DNA. This suggests that TOX-box may be involved in recognition, perhaps stabilization, of specific distorted DNA structures, rather than inducing them.

TOX may be targeted by recognizing structural features of chromatin or alternatively by binding to other proteins. Outside of the DNA-binding domains, the N-terminal domains of family members are the next most similar, and it is believed the N-domain provides transactivation activity. The C-terminal domains of the family members are quite distinct and studies suggest C-domains primarily function as interaction domains (6). The C-terminal domain of TOX3 particularly stands out from the rest of the family, as it is glutamine-rich.

The biological function of TOX-family members in vivo has not been well-characterized. TOX expression is tissue- and stage-specific, although it is not T-cell specific. Highest level of expression is observed in the thymus, with markedly reduced expression in peripheral lymphoid tissues (2). Recently, expression of TOX3 has been reported to link calcium signaling to c-fos regulation in isolated neurons (6). And TOX2 has been reported to be expressed in rat ovarian granulosa cells (7) and mouse retina (8). In addition, the inventors found expression of Tox4 mRNA fairly widespread (5). Overall, it appears that despite some overlap in tissue expression, different TOX family members may play greater or lesser roles in specific tissues. The inventors previously discovered that even in the mouse brain, where Tox and Tox3 mRNA are both expressed, they have non-overlapping patterns of expression.

Based on several key observations, the inventors have discovered that TOX3 (also known as TNRC9) has a specific role in breast cancer pathology, functioning through a novel mechanism of ER activation that is independent of estrogen E2 activation, and inducing expression of several key genes, such as TFF 1 and CXCR4. Initial focus on TOX3 was guided by gene expression studies comparing primary breast tumors from patients that were lymph node negative at the time of diagnosis, but that had experienced relapse either to bone or to other parts of the body (10). Among the genes found to be upregulated in tumors that metastasized to bone was TOX3. More recently, two articles examined genome-wide association studies (GWAS) to identify breast cancer susceptibility loci, in particular searching for common low-penetrance alleles that would be associated with disease (11, 12). Both articles reported that SNPs linked to TOX3 were associated with increased breast cancer risk. Increased disease risk appears to be most associated with estrogen receptor (ER) positive tumors (11, 13). Estrogen positivity (ER⁺) is a strong histopathological predictor of bone metastases, yielding a link between the two studies above (14). Among a European population the risk allele was present in a homozygous state at a 7% frequency and imparted a 1.64 greater disease risk (11). Among a small cohort of patients with familial breast cancer without BRCA1 and BRCA2 mutations, homozygotes for the TOX3 minor allele had a 2.4-fold increased cancer risk (15). However, TOX3 has not been associated with increased risk of ovarian cancer (17), suggesting the potential for tissue specificity in its mode of action.

A retrospective study of microarray data reported that by ANOVA analysis TOX3 mRNA was upregulated in luminal A, luminal B, and ErbB2+molecular subtypes of breast cancer tumors and downregulated in basal-like tumors (18), suggesting that TOX3 may not only play a biologically-relevant role in certain tumors, but that expression may also have some value as a biomarker. However, only statistical analysis was reported and not quantitative data on levels of expression. In another analysis of breast cancer patients, individuals homozygous for the TOX3 locus variant were more likely to be diagnosed before the age of 60 than those not homozygous for the TOX3 locus variant (19). Interestingly, minor allele frequencies for the TOX3-associated SNP were elevated among 40 human breast cancer cell lines (20). Surprisingly, however, there was no correlation between the allele and actual expression of TOX3 mRNA, although the range of expression levels for TOX3 among individual cell lines was quite broad in this report. According to particular aspects, a lack of an association between haplotype and TOX3 expression in these cell lines is due to TOX3's role during induction, but not maintenance of tumors or additional changes in these cell lines as a result of extensive propagation in culture.

As mentioned above, BRCA1 or BRCA2 mutations can impart a very high risk for breast cancer. Interestingly, even among BRCA1 and BRCA2 mutation carriers, the minor allele SNP linked to TOX3 can impart an increased risk for disease, particularly for BRCA2 mutation carriers (15, 21). This highlights the potential for additive or synergistic effects of disease susceptibility loci. In addition, the fact that cancers with BRCA2 mutations are more likely to be estrogen receptor positive than those with BRCA1 mutations (22), is also consistent with the stronger association of TOX3 variation with estrogen receptor positive (ER⁺) disease. At least in a cell line, however, expression of TOX3 itself does not appear to be estrogen E2 responsive (23).

Several common variants that impart moderately increased breast cancer risk have been identified using GWAS, including SNPs upstream of TOX3 locus, suggesting the possibility of an area of open chromatin-regulation (11, 12). These studies are independently verified in numerous other studies, but also have been associated with other features, such as BRCA1 and BRCA2 mutation carriers, triple-negative breast cancer, and risk of breast cancer in men (39-41). These GWAS reports, while highly informative, do not identify a clear biological impact associated with the presence of an SNP upstream of TOX marks changes in TOX3 expression. Such questions would be better answered with a clear understanding of the level of TOX3 expression in normal mammary epithelium, and potential roles in such tissues (e.g. TOX-like activity to influence cell differentiation or migration). Likewise, if TOX3 is expressed in breast tumors, it would be of great interest to assess the biological impact, particularly as it relates to cancer pathogenesis and tumor formation. Based on the above studies, the inventors believe that TOX3 plays a role in breast cancer and has profound effects on regulation of cellular activity during initiation, maintenance, or spread of cancer.

One embodiment of the present invention includes detection of the involvement of TOX3 in breast cancer. In different embodiments, this includes molecular subtypes such as luminal A, luminal B, luminal C, molecular-apocrine, basal, or normal-breast-like cancers. In another embodiment, the subset of breast cancer is ER⁺Her2⁺, ER⁺Her2⁻, ER⁻Her2⁺, and ER⁻Her2⁻. In another embodiment, high levels of TOX3 expression, such as being in the upper 15% of measurements compared to a reference or panel of reference samples, displays clinical features that differ from lower levels of TOX3 expression. In another embodiment, TOX3 induces expression of TFF1, TFF3, AGR2, SCUBE2, CEACAM6, TSPAN1 or CXCR4. In another embodiment, TOX3 induces expression of TFF1, TFF3, AGR2, SCUBE2, CEACAM6, TSPAN1 or CXCR4 occurs independent of estrogen E2 binding to estrogen receptor α (ESR).

In another embodiment, the present invention provides a method of reducing the likelihood of the development of cancer in a subject, including providing a composition comprising a TOX3 modulator, and administering the TOX3 modulator to the subject in an amount that is sufficient to reduce or inhibit TOX3 function and/or expression in the cancer cells and thereby inhibit the development of cancer. In another embodiment, the TOX3 modulator is a small interfering RNA, an antisense molecule, antibody, antibody fragment, polypeptide, peptide or a small molecule. In other embodiments, TOX3 modulators may serve to modulate the function of TOX3 activity, such as expression of genes induced by TOX3 expression, some examples including TFF1, TFF3, AGR2, SCUBE2, CEACAM6, TSPAN1 or CXCR4. In another embodiment, the cancer is breast cancer. In another embodiment, the cancer is a subset of breast cancer. In another embodiment, the subset of breast cancer is luminal A, luminal B, luminal C, molecular-apocrine, basal, or normal-breast-like cancer. In another embodiment, the subset of breast cancer is ER⁺Her2⁺, ER⁺Her2⁻, ER⁻Her2⁺, and ER⁻Her2⁻. In another embodiment, the subset of breast cancer is tamoxifen resistant. In another embodiment, the subset of breast cancer is fulvestant sensitive. In another embodiment, the breast cancer is metastatic.

In another embodiment, the present invention provides a method of determining the subtype of cancer in a subject, including obtaining a test sample from a subject, determining the expression level of at least one biomarker in the test sample, comparing the expression level of the at least one biomarker in the test sample with the expression level the at least one biomarker in a reference sample from a healthy individual, and determining that the subject has a particular subtype of cancer based on the level of expression of the at least one biomarker in the test sample compared to the level of expression of the at least one biomarker in the reference sample from the healthy individual. In another embodiment, the test sample comprises a tissue or a cell. In another embodiment, the at least one biomarker is TOX3, TFF1, TFF3, AGR2, SCUBE2, CEACAM6, TSPAN1 and/or CXCR4. In another embodiment, the level of TOX3 is measured and classified as high (TOX3^{hi}) expression as being in the upper 15% of measurements compared to a reference or panel of reference samples. In different embodiments, biomarkers include CD24, CD29, CD61, Lin, estrogen receptor a (ESR), FOXA1 and SCA-1. In one embodiment, estrogen receptor a (ESR) activation is measured via phosphorylation, such as phosphorylation at serine 118. In another embodiment, determining the expression level of the at least one biomarker comprises analyzing the transcription level of the at least one biomarker or analyzing the protein level of the at least one biomarker. In another embodiment, the cancer is breast cancer. In another embodiment, the cancer is a subset of breast cancer. In another embodiment, the subset of breast cancer is luminal A, luminal B, luminal C, molecular-apocrine, basal, or normal-breast-like cancer. In another embodiment, the subset of breast cancer is ER⁺Her2⁺, ER⁺Her2⁻, ER⁻Her2⁺, and ER⁻Her2⁻. In another embodiment, the subset of breast cancer is tamoxifen resistant. In another embodiment, the subset of breast cancer is fulvestant sensitive. In another embodiment, the breast cancer is metastatic.

In another embodiment, the present invention provides a method of determining an increased susceptibility of a subject to cancer, including obtaining a test sample from the subject, determining the expression level of at least one biomarker in the test sample, comparing the expression level of the at least one biomarker in the test sample with the expression level of the at least one biomarker in a reference sample from a healthy individual, and determining that the subject has an increased susceptibility to cancer based on the level of expression of the at least one biomarker in the test sample compared to the level of expression of the at least one biomarker in the reference sample from the healthy individual. In another embodiment, the sample comprises a tissue or a cell. In another embodiment, the at least one biomarker is TOX3, TFF1, TFF3, AGR2, SCUBE2, CEACAM6, TSPAN1 and/or CXCR4. In another embodiment, the level of TOX3 is measured and classified as high (TOX3^{hi}) expression as being in the upper 15% of measurements compared to a reference or panel of reference samples. In different embodiments, biomarkers include CD24, CD29, CD61, Lin, estrogen receptor a (ESR), FOXA1 and SCA-1. In another embodiment, determining the expression level of the at least one biomarker comprises analyzing the transcription level of the at least one biomarker or analyzing the protein level of the at least one biomarker. In another embodiment, the cancer is breast cancer. In another embodiment, the cancer is a subset of breast cancer. In another embodiment, the subset of breast cancer is luminal A, luminal B, luminal C, molecular-apocrine, basal, or normal-breast-like cancer. In another embodiment, the subset of breast cancer is ER⁺Her2⁺, ER⁺Her2⁻, ER⁻Her2⁺, and ER^{-Her}2⁻. In another embodiment, the subset of breast cancer is tamoxifen resistant. In another embodiment, the subset of breast cancer is fulvestant sensitive. In another embodiment, the breast cancer is metastatic.

In another embodiment, the present invention provides a method of treating cancer in a subject, including providing a composition including a TOX3 modulator, and administering the composition to the subject in an amount sufficient to reduce or inhibit TOX3 function and/or expression in the subject's cancer cells, whereby the cancer is treated. In another embodiment, the TOX3 modulator is a small interfering RNA, an antisense molecule, antibody, antibody fragment, polypeptide, or a small molecule. In other embodiments, TOX3 modulators may serve modulate the function of TOX3 activity, such as expression of genes induced by TOX3 expression, some examples including TFF1, TFF3, AGR2, SCUBE2, CEACAM6, TSPAN1 or CXCR4. In another embodiment, the cancer is breast cancer. In another embodiment, the cancer is a subset of breast cancer. In another embodiment, the subset of breast cancer is luminal A, luminal B, luminal C, molecular-apocrine, basal, or normal-breast-like cancer. In another embodiment, the subset of breast cancer is ER⁺Her2⁺, ER⁺Her2⁻, ER⁻Her2⁺, and ER⁻Her2⁻. In another embodiment, the subset of breast cancer is tamoxifen resistant. In another embodiment, the subset of breast cancer is fulvestant sensitive. In another embodiment, the breast cancer is metastatic.

In another embodiment, the present invention provides a kit, including a composition comprising a TOX3 modulator, and instructions for the use of the composition for reducing the likelihood of the development of cancer in a subject. In another embodiment, the TOX3 modulator is an small interfering RNA, an antisense molecule, antibody, antibody fragment, polypeptide, or a small molecule.

In another embodiment, the present invention provides a kit, including a composition, and instructions for the use of the composition for determining the subtype of cancer in a subject. In another embodiment, the composition is a nucleotide, antibody, or a small molecule.

In another embodiment, the present invention provides a kit, including a composition, and instructions for the use of the composition for determining an increased susceptibility of a subject to cancer. In another embodiment, the TOX3 modulator is a nucleotide, antibody, or a small molecule.

In another embodiment, the present invention provides a kit, including a composition comprising a TOX3 modulator, and instructions for the use of the composition for treating cancer in a subject. In another embodiment, the TOX3 modulator is small interfering RNA, an antisense molecule, antibody, antibody fragment, polypeptide, or a small molecule.

In various embodiments, kits for prognosis, diagnosis, treatment, subtype classification, or reducing likelihood of disease and/or condition, contain an assemblage of materials or components, including at least one of the inventive compositions. In different embodiments, the compositions include modulators of TOX3 function and/or expression. Inventive modulators include, but are not limited to, small interfering RNAs, antisense molecules, antibodies or antibody fragments, proteins, polypeptides, peptides, as well as small molecules. The exact nature of the components configured in the inventive kit depends on its intended purpose. For example, some embodiments are configured for the purpose of treating cancer patients. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals. Instructions for use may be included in the kit. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). The packaging material generally has an external label which indicates the contents and/or purpose of the kit and/or its components.

In another embodiment, the present invention provides a pharmaceutical composition, including, a therapeutically effective amount of a TOX3 modulator, or a pharmaceutical equivalent, derivative, analog, and/or salt thereof, and a pharmaceutically acceptable carrier.

In other embodiments, a therapeutic agent may be a TOX 3 modulator, which reduces or eliminate TOX3 function and/or expression in cells. TOX3 modulators and compositions comprising one or more TOX3 modulator as well as methods that employ these inventive inhibitors in in vivo, ex vivo, and in vitro applications where it is advantageous to reduce or eliminate the expression or activity of TOX3 or a functionally-downstream molecule. In other embodiments, TOX3 modulators may serve modulate the function of TOX3 activity, such as expression of genes induced by TOX3 expression, some examples including TFF1, TFF3, AGR2, SCUBE2, CEACAM6, TSPAN1 or CXCR4. In various embodiments, TOX3 modulators may include tamoxifen, fulvestant, getifinib, erlotinib, Her2 inhibitors, trastuzumab, lapatinib imanitib mesylate, mTor inhibitors, farnesyl transferase inhibitors, antiangiogenic agents, anti-estrogens, aromastase inhibitors, TOX3 modulators may also find use in other diseases of hyperproliferation. In various embodiments, TOX3 modulators may supplement the activities of tamoxifen, Fulvestant, getifinib, erlotinib, Her2 inhibitors, trastuzumab, lapatinib imanitib mesylate, mTor inhibitors, farnesyl transferase inhibitors, antiangiogenic agents, anti-estrogens, and aromastase inhibitors.

In different embodiments, pharmaceutical compositions that include a TOX3 modulator may be administered parenterally, topically, orally, or locally for therapeutic treatment. In different embodiments, compositions are administered orally or parenterally, i.e., intravenously, intraperitoneally, intradermally, or intramuscularly. In different embodiments, the pharmaceutical compositions can include one or more TOX3 modulator and may further include a pharmaceutically-acceptable carrier or excipient. In different embodiments, a variety of aqueous carriers may be used, e.g., water, buffered water, 0.4% saline, 0.3% glycine, and the like, and may include other proteins for enhanced stability, such as albumin, lipoprotein, globulin, etc., subjected to mild chemical modifications or the like. In different embodiments, TOX3 modulators useful in the treatment or prevention of disease in mammals will often be prepared substantially free of naturally-occurring immunoglobulins or other biological molecules. In one embodiment, TOX3 modulators will also exhibit minimal toxicity when administered to a mammal.

In different embodiments, the compositions of the invention may be sterilized by conventional, well-known sterilization techniques. The resulting solutions may be packaged for use or filtered under aseptic conditions and lyophilized, the lyophilized preparation being combined with a sterile solution prior to administration. In different embodiments, the compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

In various embodiments, the selection of the appropriate method for administering TOX3 modulators of the present invention will depend on the nature of the application envisioned as well as the nature of the TOX3 modulator. In different embodiments, the precise methodology for administering a TOX3 modulator will depend upon whether it is small interfering RNA, an antisense molecule, a protein and/or peptide, an antibody or antibody fragment, or a small molecule. In different embodiments, the TOX3 modulator will be used to regulate tumor cell initiation, growth, invasion, or metastasis, or as an adjunct to other cancer therapeutics. In other embodiments, TOX3 modulators may serve modulate the function of TOX3 activity, such as expression of genes induced by TOX3 expression, some examples including TFF1, TFF3, AGR2, SCUBE2, CEACAM6, TSPAN1 or CXCR4.

In different embodiments, a variety of methods are available in the art for the administration of small interfering RNA, antisense molecules, gene delivery for transgene expression or gene knockout. In different embodiments, this includes gene delivery techniques, including both viral and non-viral based methods as well as liposome-mediated delivery methods. In certain embodiments, gene delivery methodologies will be effective to, for example, reduce tumor cell proliferation, or supplement radiation and/or chemotherapeutic treatment of tumors. See, Wheldon, T. E. et al., Radiother Oncol 48(1):5-13 (1998) (gene delivery methodologies for enhancement of fractionated radiotherapy). In certain embodiments, gene delivery methodology may be used to directly knock-out endogenous TOX3 within tumor cells. In one embodiment, the TOX3 gene may be targeted by transfection of a gene delivery vector carrying a TOX3 modulator. In different embodiments, preferential transfection into or expression within tumor cells may be achieved through use of a tissue-specific or cell cycle-specific promoter, such as, e.g., promoters for BRCA2 or for immunoglobulin genes (Vile, R. G. et al., Cancer Res. 53:962-967 (1993) and Vile, R. G., Semin. Cancer Biol. 5:437-443 (1994)) or through the use of trophic viruses that are confined to particular organs or structures, such as, e.g., a replication selective and neurotrophic virus that can only infect proliferating cells in the central nervous system. In different embodiments, substantial therapeutic benefit may be achieved despite transfection efficiencies significantly less than 100%, transient retention of the transfected inhibitor, and/or existence of a subpopulation of target cells refractory to therapy. In different embodiments, TOX3 within the tumor cells is preferentially modulated to achieve therapeutic benefit. In different embodiments, this is accomplished by transfecting a gene expressing a TOX3 inhibitor, a TOX3 cognate small interfering RNA, a TOX3 antisense molecule, a TOX3 gene-specific repressor, or an inhibitor of the protein product of the TOX3 gene.

In different embodiments, peptides or polypeptides may be used for direct binding to a protein, such as a soluble receptor, or as antigen for the generation of antigen-specific antibodies. In one example, the present invention provides for a peptide which is capable of binding TOX3. The peptide may be prepared by chemical synthesis or biochemical synthesis using Escherichia coli or the like. Methods well-known in those skilled in the art may be used for the synthesis. In addition to the baculovirus expression system, other suitable bacterial or yeast expression systems may be employed for the expression of TOX3 protein or polypeptides thereof. As with the baculovirus system, it may be advantageous to utilize one of the commercially-available affinity tags to facilitate purification prior to inoculation of the animals. Thus, the TOX3 cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998). In other examples, the peptide is capable of binding to TFF1, TFF3, AGR2, SCUBE2, CEACAM6, TSPAN1 and/or CXCR4.

In another embodiment, the present invention provides methods for using TOX3 peptides or polypeptides to produce antibodies or similar TOX3 binding proteins. In different embodiments, TOX3 peptides are useful in producing antibodies can be made from the TOX3 polypeptide of SEQ ID NO:1 containing amino acids from about position 1 to about 576, from about position 1 to about 238, from about position 1 to about 150, from about position 2 to about 238, and from about position 2 to about 150. In different embodiments, TOX3 peptides useful in producing antibodies can be made from the N-terminal portion of TOX3 polypeptide (SEQ ID NO:2) containing between 5 to 10 consecutive amino acids, containing between 5 to 15 consecutive amino acids, containing between 5 to 20 consecutive amino acids, containing between 5 to 25 consecutive amino acids, containing between 5 to 30 consecutive amino acids, containing between 5 to 35 consecutive amino acids, containing between 5 to 40 consecutive amino acids, containing between 5 to 45 consecutive amino acids, containing between 5 to 50 consecutive amino acids, containing between 5 to 55 consecutive amino acids, containing between 5 to 60 consecutive amino acids, containing between 5 to 65 consecutive amino acids, containing between 5 to 70 consecutive amino acids, containing between 5 to 75 consecutive amino acids, containing between 5 to 80 consecutive amino acids, containing between 5 to 85 consecutive amino acids, containing between 5 to 90 consecutive amino acids, containing between 5 to 95 consecutive amino acids, containing between 5 to 100 consecutive amino acids, containing between 5 to 105 consecutive amino acids, containing between 5 to 110 consecutive amino acids, containing between 5 to 115 consecutive amino acids, containing between 5 to 120 consecutive amino acids, containing between 5 to 125 consecutive amino acids, containing between 5 to 150 consecutive amino acids, containing between 5 to 175 consecutive amino acids, containing between 5 to 200 consecutive amino acids, and containing between 5 to 238 consecutive amino acids.

In different embodiments, TOX3 peptides can be used to produce antibodies or similar TOX3 binding proteins. TOX3 peptides useful in producing antibodies can be made from the TOX3 polypeptide containing amino acids. In different embodiments, peptides of length X (in amino acids), as indicated by polypeptide positions with reference to, e.g., SEQ ID NO:1, include those corresponding to sets of consecutively overlapping peptides of length X, where the peptides within each consecutively overlapping set (corresponding to a given X value) are defined as the finite set of Z peptides from amino acid positions:

n to (n+(X−1));

where n=1,2,3, . . . (Y−(X−1));

where Y equals the length (amino acid or base pairs); where X equals the common length (in amino acid) of each peptide in the set (e.g., X=10 for a set of consecutively overlapping 10-mers); and where the number (Z) of consecutively overlapping oligomers of length X for a given sequence of length Y is equal to Y−(X−1). In different embodiments, 10-mer peptide within a sequence of length 576 amino acid residues include the following set of 576 oligomers, indicated by polypeptide positions 1-20, 2-21, 3-22, 4-23, 5-24 to 566-576. In different embodiments, each of SEQ ID NOS:1 and SEQ ID NO:2, multiple consecutively overlapping sets of peptides or modified peptides of length X, where, e.g., X=9, 10, 17, 20, 22, 23, 25, 27, 30, or 35 amino acids. In different embodiments, peptides or modified peptides of length X are those consecutively overlapping sets of oligomers corresponding to SEQ ID NOS:1 and SEQ ID NO:2.

In another embodiment, the present invention provides an assay to detect the presence and level of different biomarkers, either an allele, a transcript, or protein as they relates to certain subsets of cancers (e.g. breast cancers). In different embodiments, these biomarkers are TOX3, TFF1, TFF3, AGR2, SCUBE2, CEACAM6, TSPAN1 or CXCR4. In another embodiment, the level of TOX3 is measured and classified as high (TOX3^{hi}) expression as being in the upper 15% of measurements compared to a reference or panel of reference samples. In different embodiments, biomarkers include CD24, CD29, CD61, Lin, estrogen receptor a (ESR), FOXA1 and SCA-1. In different embodiments, TOX3, TFF1, TFF3, AGR2, SCUBE2, CEACAM6, TSPAN1 or CXCR4 are indicative of certain subsets of cancers (e.g. breast cancers). In different embodiments, these subsets of cancers include molecular-apocrine (mApo), normal-like (normL), basal-like (basL), luminal A (lumA), luminal B (lumB), and luminal C (lumC). In another embodiment, the subset of breast cancer is ER⁺Her2⁺, ER⁺Her2⁻, ER⁻Her2⁺, and ER⁻Her2⁻. In another embodiment, the subset of breast cancer is tamoxifen resistant. In another embodiment, the subset of breast cancer is fulvestant sensitive. In another embodiment, the breast cancer is metastatic. In different embodiments, the present invention provides a kit for detecting polynucleotides and/or proteins for biomarkers.

In different embodiments, the present invention provides peptides that can be used to produce antibodies or similar binding proteins to TOX3, TFF1, TFF3, AGR2, SCUBE2, CEACAM6, TSPAN1 or CXCR4. According to further embodiments, antibodies are useful to detect the presence of TOX3, TFF1, TFF3, AGR2, SCUBE2, CEACAM6, TSPAN1 or CXCR4 in cells and tissue samples. In another embodiment, antibodies are useful to measure the protein level of TOX3. In different embodiments, biomarkers include CD24, CD29, CD61, Lin, estrogen receptor a (ESR), FOXA1 and SCA-1. In various embodiments, antibodies, such as TOX3 specific antibodies, are capable of staining tissue microarrays, and distinguishing between tumor, non-tumor samples, including positive staining for tumors in tissue sections and negative staining for adjacent healthy tissue.

According to additional aspects of the various methods, kits, and pharmaceutical compositions described herein, the present invention provides for the use of nucleic acids and/or proteins to detect an increase in susceptibility to certain types of cancers, e.g. breast cancer. In different embodiments, these nucleic acids and/or proteins are specific for TOX3 TFF1, TFF3, AGR2, SCUBE2, CEACAM6, TSPAN1 CXCR4, CD24, CD29, CD61, Lin, estrogen receptor a (ESR), FOXA1 and/or SCA-1.

Polynucleic Acid Detection.

There are many techniques readily available in the field for detecting the presence, absence, and/or level of an allele, transcript, or other biomarker, including mRNA microarrays. For example, enzymatic amplification of nucleic acid from an individual may be used to obtain nucleic acid for subsequent analysis (e.g., polymerase chain reaction (PCR), endpoint and quantitative reverse transcriptase-PCR (RT PCR)). The presence or absence of allele, transcript or other biomarker may also be determined directly from the individual's nucleic acid without enzymatic amplification.

Analysis of the nucleic acid from an individual, whether amplified or not, may be performed using any of various techniques. Useful techniques include, without limitation, PCR-based analysis, sequence analysis, and electrophoretic analysis.

Protein Detection and/or Biomarker Detection.

There are many techniques readily available in the field for detecting the presence or absence of polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method, or interferometry).

Similarly, there are any numbers of techniques that may be employed to isolate and/or fractionate biomarkers. For example, a biomarker may be captured using biospecific capture reagents, such as antibodies, aptamers, or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer, and hybrids of these.

Alternatively, for example, the presence of biomarkers such as polypeptides may be detected using traditional immunoassay techniques. Immunoassay requires biospecific capture reagents, such as antibodies, to capture the analytes. The assay may be designed to specifically distinguish protein and modified forms of protein, which can be done by employing a sandwich assay in which a first antibody captures more than one form and a second distinctly-labeled antibody specifically binds and provide distinct detection of the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or blood that may interfere with detection. Fractionation may include platelet isolation from other blood components, sub-cellular fractionation of platelet components, and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface to which a capture reagent (also called an adsorbent or affinity reagent) is attached. Frequently, the surface of a biochip comprises a plurality of addressable locations, each of which has the capture reagent bound there.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Microarray Analysis of TOX3 Gene Expression

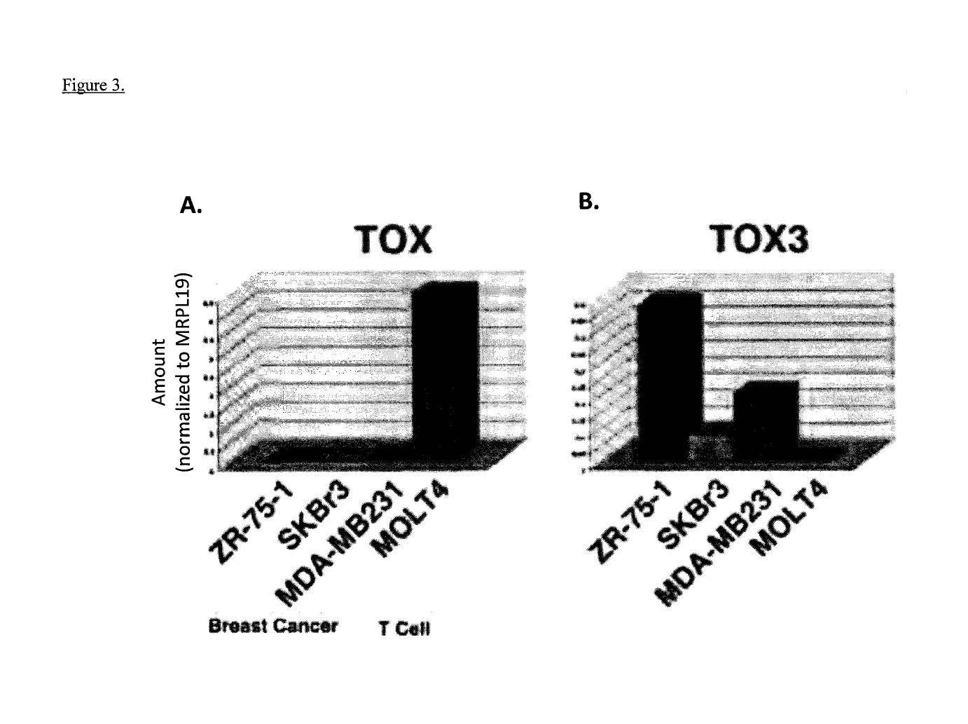

The inventors examined existing microarray data and determined the presence of TOX3 transcript in breast cancer cell lines and tumors. This analysis is a critical first step to take these studies beyond SNP associations. It should be noted that the expression of TOX3 appears to be relatively restricted in normal tissues, with highest levels of expression in fetal brain (BioGPS; http://biogps.gnf.org). As shown in FIG. 2, the inventors analyzed two microarray studies (24, 25), organizing heat maps based on expression of TOX3 (UCSC Cancer Genomics Browser).

Interestingly, expression of TOX3 dramatically subsets both cell lines and tumors into high (FIG. 2A) and low expressers (FIG. 2B). This suggests that expression levels of TOX3 may act as a novel marker to subset tumors. The inventors compared this same ordered data set with expression of other select genes, including TOX itself. Results from this analysis include a number of interesting and surprising points. In both tumors and cell lines there appears to be an inverse correlation between expression of TOX and TOX3. According to particular aspects, based on the near identity of the DNA-binding domains and the differences elsewhere in these proteins, these two family members in essence act as dominant negative mutants of each other (i.e. compete for DNA binding but have different functions). In addition, there is a positive, although not absolute, correlation between estrogen receptor (ESR1) expression and TOX3 expression. This would be consistent with a more dominant role for TOX3 in ER⁺disease, as discussed above.

The transcription factor GATA3 is often co-expressed with estrogen receptor alpha in breast cancer cells and is one molecular marker of the luminal A subtype of breast cancer (26, 27). In addition, there is an overall positive correlation between GATA3 and TOX3 expression in the cell lines studied. In general, there is a positive correlation between TOX3 expression and ErbB2 and GRB7 expression (the latter analyzed for tumors). GRB7 is an SH2-domain adaptor protein that binds to receptor tyrosine kinases and is genetically linked to the ErbB2 (HER2/neu) proto-oncogene. ErbB2 and GRB7 are commonly co-amplified in breast cancers. Interestingly, in an analysis that examined the expression of CD44 and TOX3, the inventors discovered that there was an inverse correlation between expression of CD44 and TOX3, which is consistent with poor expression of TOX3 in the basal subtype (CD44 has been suggested as one marker for cancer stem cells and expression of CD44 may be associated with basal-like disease (28)). Finally, the inventors found no association with c-fos, distinguishing the possible action of TOX3 in breast cancer from that observed in neurons (6). There is certainly cellular heterogeneity within all these samples and thus expression of these genes on a per cell basis is unknown.

Further meta-analysis of the microarray data indicated that several genes are significantly upregulated, as associated with high and low levels of TOX3 expression. For TOX3^{hi} expression, these genes include TFF1, TFF3, AGR2, SCUBE2, CEACAM6, and TSPAN (Table 1).

TABLE 1
Upregulated gene expression in TOX3^{hi}

| Gene | Fold Ratio (TOX3hi vs. TOX3lo) | P-value (T Test) |
|---|---|---|
| TFF1 | 4.6-fold up | 2.77E−36 |
| TFF3 | 4.3-fold up | 2.30E−28 |
| AGR2 | 3.1-fold up | 2.88E−45 |
| SCUBE2 | 1.8-fold up | 6.24E−09 |
| CEACAM6 | 4.9-fold up | 1.21E−21 |
| TSPAN1 | 4.4-fold up | 4.69E−29 |

### Example 2

### TOX3 Gene Expression in Breast Cancer Cell Lines and Tumors

For initial TOX3 expression studies, the inventors examined directly whether TOX3 is expressed in breast cancer cells using quantitative RT-PCR. MOLT4, an oft-studied human acute lymphoblastic leukemia cell line, highly expressed TOX, but not TOX3 (FIG. 2). In contrast, three oft-studied breast cancer cell lines expressed TOX3 to various levels (FIG. 3B), but did not express TOX (FIG. 3A). Interestingly, ZR75-1, the highest expresser, is an ER⁺ luminal subtype of breast cancer cell (24). These results are consistent with the role of TOX in the immune system and the role of TOX3 in breast cancer. Moreover, since the tissue microenvironment can greatly influence cancer cells and microarray analysis of tumors includes a heterogeneous population of cells, these result confirm expression of TOX3 by the cancer cell itself.

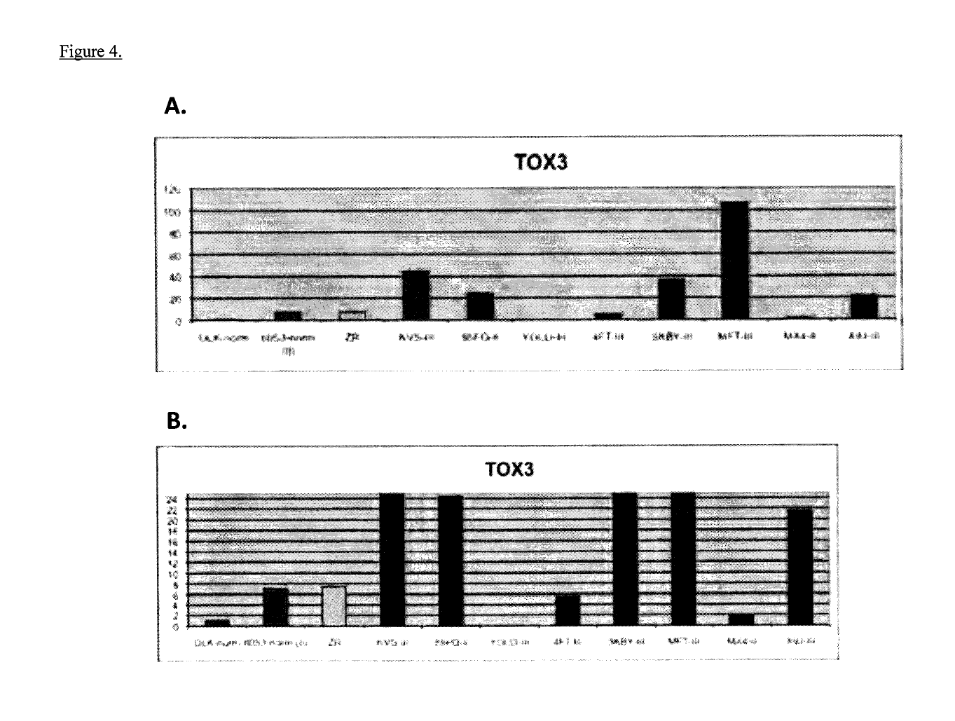

TOX3 expression was further analyzed by qRT-PCR in RNA derived from 8 breast cancer tumors and 2 normal breast tissue samples; all samples were obtained from a commercial source. The breast cancer RNA samples were pre-selected by the following minimal criteria; the patients were (1) female, (2) White/Caucasian, and (3) had been diagnosed with estrogen receptor positive disease. All tumors were stage II or stage III infiltrating ducal carcinomas, from patients aged 41 to 78 years of age. For the two normal samples, one was from a 46-year-old patient who did not have cancer and one was derived from normal tissue from a 73-year-old patient diagnosed with stage II breast cancer. The inventors normalized the results to the sample from the non-cancer patient, arbitrarily assigning a value of 1 (FIGS. 4A and 4B). Surprisingly, the second “normal” sample had a 7-fold increase in TOX3 expression, similar to that seen in the ZR75-1 breast cancer cell line (FIG. 4B). According to certain embodiments, this result is related to the fact that this sample was derived from a cancer patient, and thus reflects an inherent variability in normal expression of this gene. Interestingly, there was great variability in expression of TOX3 among these tumor samples, ranging from well below that even detected in the normal tissue to greater than 100-fold upregulated in one tumor sample (MFT) (FIG. 4A). This is reminiscent of the microarray data described above. According to certain embodiments, these expression differences correlate with TOX3 locus allelic differences. Intriguingly, though, only two of the eight patients had reported that their mothers also had breast cancer, and these were among the top three expressers of TOX3 (samples MFT and SKBY).

### Example 3

### Predominant TOX3 Transcript in Breast Cancer Cell Lines and Tumors

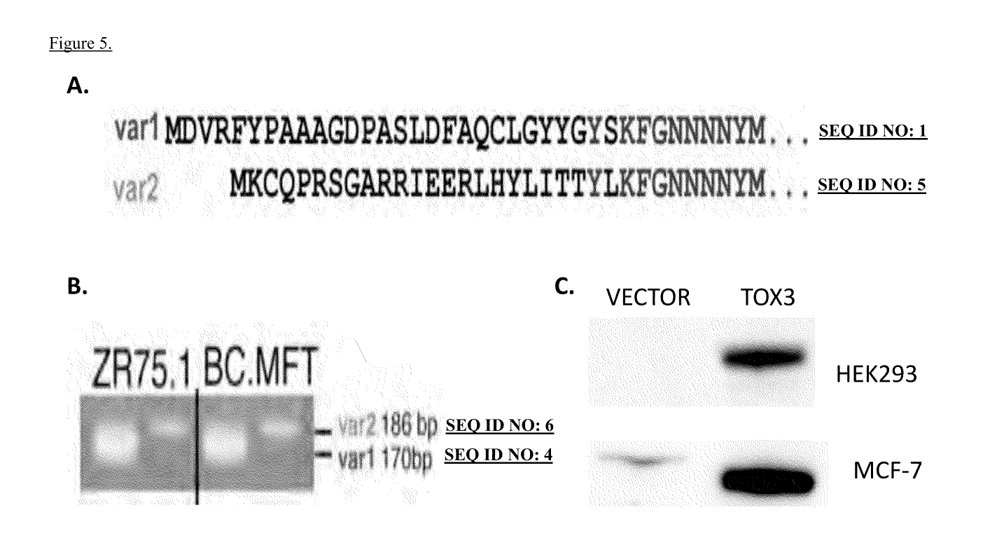

Two TOX3 transcripts have been reported that encode different N-terminal ends of the protein (FIG. 5A). The shorter variant 2 includes an alternative exon within the otherwise first intron of the TOX3 locus. A common 3′ primer and distinct 5′ primers that allowed the two transcripts to be distinguished were designed. Specific detection of variant 1 results in amplification of a 170 base pair fragment, whereas variant 2 results in amplification of a 186 base pair fragment. These were used in end point RT-PCR on RNA derived from the ZR75.1 cell line and the MFT breast cancer tumor (FIG. 5B). Results indicated that variant 1 is the predominant transcript, even in primary tumor cells (FIG. 5B). Thus, the in vivo work will focus on this form of the protein.

### Example 4

### Transcriptome Analysis of Tumors with High TOX3 Expression

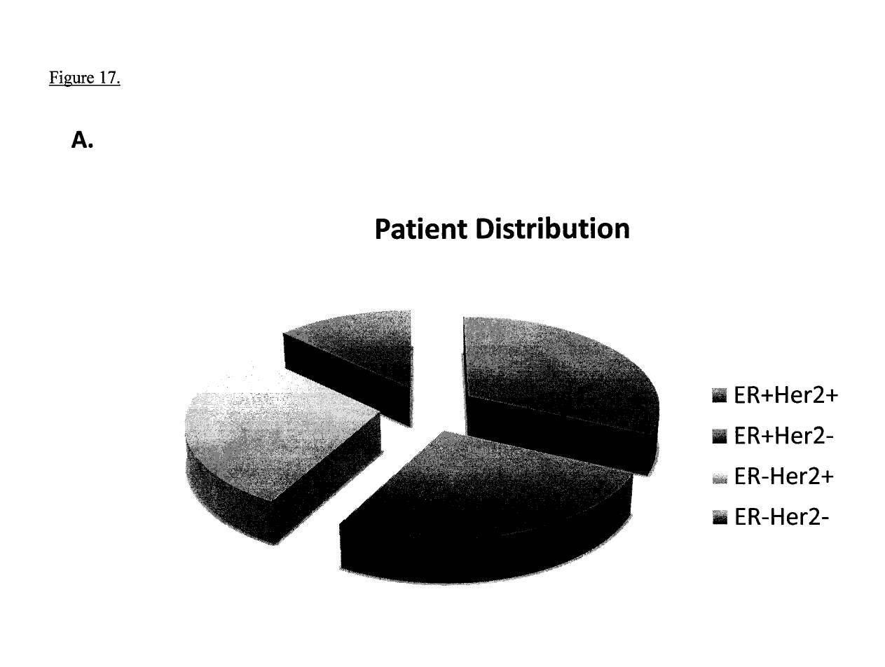

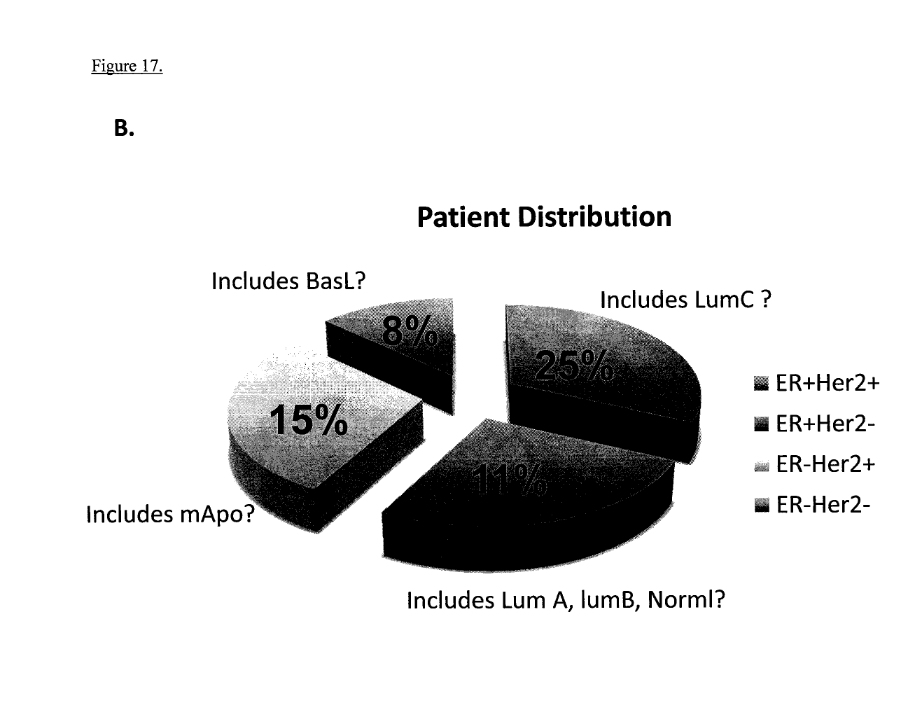

Breast cancer tumors that highly express TOX3 were analyzed using transcriptome profiling. A long-standing classification of breast cancers in the clinic has provided three broad categories for classification drawn along their ER, PR and ERBB2/HER2 status. This includes: 1) ER−/PR−/HER2− tumors were defined as “triple negative”, 2) ER+/PR+/HER2− described as “luminal”, and 3) HER2+ tumors irrespective of their ER status form the third class (FIG. 17). This set of classifications, while useful, are being supplanted by more thorough and precise molecular subtype classifications.

Global expression analysis has enabled classification of molecularly-defined subsets of cancers. For breast cancer, five subtypes: luminal A (lumA), luminal B (lumB), ErbB2-enriched, basal (basL), and normal-breast-like (normL), were initially proposed based on gene expression clusters that were relatively stable over time and have some clinical correlations (26, 29). More recently, the ErbB2 has been organized into molecular-apocrine (mApo) and luminal C (lumC) subtypes, thereby providing an enhanced classification for six molecular sub-types. A rough overlap between this molecular subtype classification and existing tripartite clinical classification is shown (FIG. 17B). However, molecular and clinical classifications belie the complexity and heterogeneity of the disease even within subtypes. Indeed, a subsequent large real-time RT-PCR analysis proposed twelve disease subtypes, based on expression of 47 genes (30). In addition, in terms of individual genes, this classification does not necessarily separate out important functional components of tumor formation or maintenance, which may be shared among subtypes, from useful but not necessarily causative biomarkers. As elaborated above, the inventors proposes to identify TOX3 as a disease susceptibility locus.

The data indicates that high level of expression of TOX3 may not fit neatly into otherwise defined subtypes. To address this issue, global gene expression analysis by microarray was performed to compare tumors with very high TOX3 expression (TOX3^{hi}) and tumors with very low TOX3 (TOX3^{lo}) expression. While a small number of samples cannot alone be used to define a new molecular subtype, the inventors can use this data to narrow the number of genes that may be proximal gene targets of TOX3. In addition, the data for expression of genes that have been previously used to define subtypes is examined to see how these samples fall within those groups. While much microarray analysis is dependent on calls of relatively modest changes in gene expression, the inventors will take a much more stringent approach. As TOX3 is a transcriptional regulator, the inventors have implemented a focused approach by first labeling genes that are highly expressed in the absence or presence of TOX3, and further investigating differential expression levels of that may result from TOX3 function.

Those genes whose expression is shared in TOX3^{hi} cells (or show correlation with levels of TOX3 expression) and whose expression is absent or low in tumors that express little TOX3 are first examined. For that reason, the inventors' quantitative data on TOX3, measured through qRT-PCR, is used as the initial resource for analysis. Then, genome annotation for related molecules provides a supplementary resource to evaluate known regulators of cell growth, survival, differentiation, or gene regulation, thereby identifying good candidates for follow-up as potential TOX3 gene targets in the context of breast cancer.

### Example 5

### Molecular and Cellular Effects of TOX3 Expression

The molecular and cellular effects of manipulating TOX3 expression in breast cancer are examined using the ZR75-1 cell line that expresses TOX3 in a complementary approach. ZR75-1 cells previously have been reported to be transfectable and susceptible to siRNA-mediated knockdown (31). Thus using siRNA-mediated TOX3 knockdown the inventors can determine directly whether expression of candidate genes is modulated by expression of TOX3. Therefore, this analysis is accomplished on a global level via microarray, and is used as comparison with the data set obtained from primary tumor samples as above. Importantly, the inventors determined if knockdown of TOX3 alters the growth, adhesion, or morphologic characteristics of this cell line, including the migration and invasion properties of the cells as assessed in vitro (31). Similarly, TOX3 is over-expressed in these cells to test for complementary changes in cellular behavior or gene expression. If differences are detected upon loss of TOX3 in these cells, then the cells can be used to test if TOX might act as a dominant negative of TOX3 function in this cellular context. This provides the basis for thinking of ways to manipulate TOX3 activity, rather than expression. Together, these studies represent a powerful approach to identify gene targets of TOX3 and correlate that with cell behavior, as well as expression in primary tumors. This result indicates that TOX3 activation can be manipulated by overexpressing TOX in breast cancer tumors.

### Example 6

### Production of Anti-TOX3 Antibodies

A rabbit polyclonal antibody against TOX was produced utilizing recent advances in production of rabbit monoclonal antibodies (rabbit antibodies are often of much higher affinity than those produced in other species). This anti-TOX3 antibody was named AJ33.

An anti-TOX3 antibody, such as AJ33, is invaluable in characterizing expression of TOX3 in breast cancer tumors. There are large numbers of well-characterized tissue arrays available for breast cancer (i.e. of known histological appearance and grade, metastatic properties, hormone receptor expression, and Her2 expression), some including adjacent normal tissue. One example is tissue microarrays (TMA) provided through NIH cancer diagnosis program, which includes both progression and prognostic case sets, and different stage I-III prognostic samples. Using these TMAs, one may determine the expression pattern of TOX in tumors at the protein level, as analogous to the molecular subtyping approach. Since these are fixed samples, initially an anti-peptide antibody that is likely to recognize denatured protein (and thus will also be useful for immunoblotting) is used.

In vivo studies require detection of an antibody that binds to the native form of TOX3 In addition. For these studies, the inventors utilized the N-terminal regions of the protein as a antigenic peptide source, which allows for discrimination from other family members, avoiding the highly conserved DNA binding domain and the Q-rich C-terminal domain. The resulting TOX3 rabbit polyclonal antibody, named AJ33, is capable of specifically detecting TOX3 in HEK293 and MCF-7 cells transfected with TOX3 vector (FIG. 5C). Vector control demonstrates that HEK 293 cells do not endogenously express TOX3, while MCF-7 express a minimal level of native TOX3 protein (FIG. 5C).

### Example 7

### Analysis of TOX3 Protein Expression

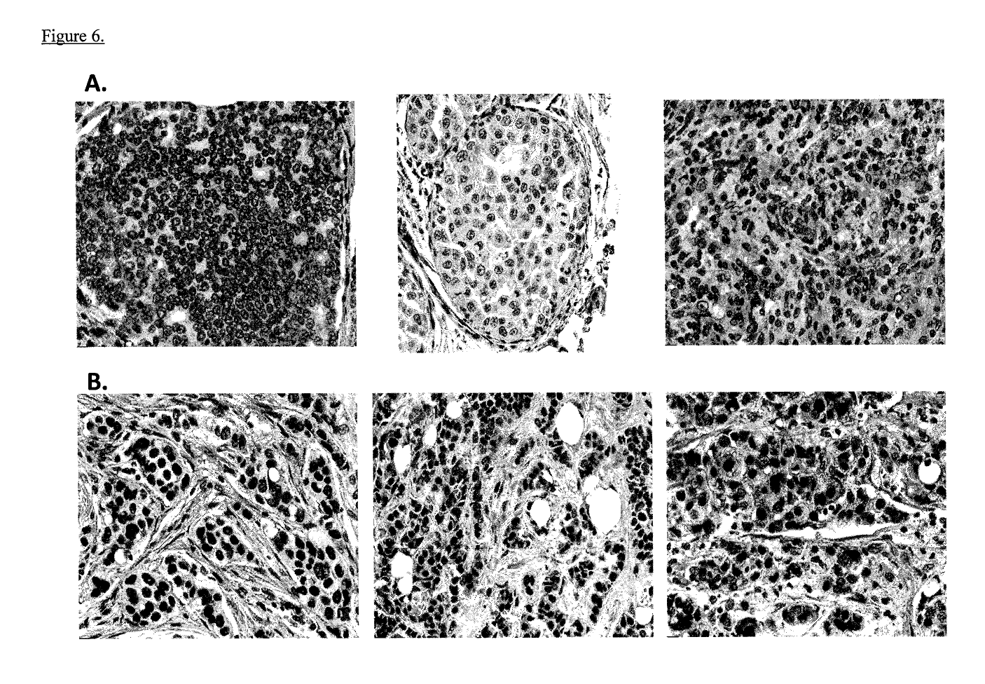

TOX3 protein levels in breast cancer tumors can be analyzed using anti-TOX3 antibodies, such as AJ33. These studies complement gene expression studies, in order to understand the distinction between tumors that express or do not express TOX3. Indeed, protein expression profiling has also been undertaken as a method to subtype breast cancers (32). Analysis of tissue arrays that include normal breast tissue as well as normal tumor-adjacent breast tissue is also of particular interest to understand interactions between cell and tissue compartments in various organ structures in the mammary gland. Staining for TOX3 in breast tissue microarray using AJ33 demonstrates that normal, non-tumor samples not show TOX3 staining (FIG. 6A). By contrast, AJ33 detection of TOX3 results in dark compact intracellular positive staining in various tumor samples (FIG. 6B). Anti-TOX3 antibody, AJ33, is capable of distinguishing positive TOX3 staining in tumor cells, compared to normal breast tissue. This platform provides a means to quantify of TOX3 expression levels that may variable both in tumor cells and normal breast tissue.

Establishing the quantification of TOX3 expression, both at the gene transcript and protein level is important as initial qRT-PCR studies have shown highly variable levels of TOX3 expression across normal tissue, tumor samples, and breast cancer cell lines. For example, TOX3 gene transcript (i.e., mRNA) levels were shown to be variable even between two ostensibly normal breast tissue samples, and with even higher expression from tissue derived from breast cancer patient samples (FIGS. 4A and 4B). Thus, protein detection of TOX3 serves as a complementary approach to qRT-PCR studies to establish when upregulation of TOX3 can ultimately lead to tumorigenesis, and how expression of TOX3 in normal tissue adjacent to a TOX3 high-expressing cells may interact for cancer pathogenesis.

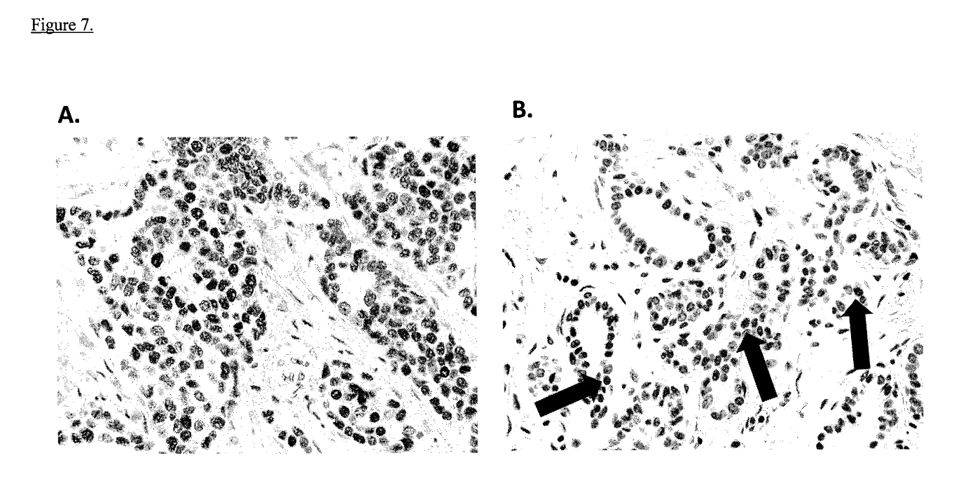

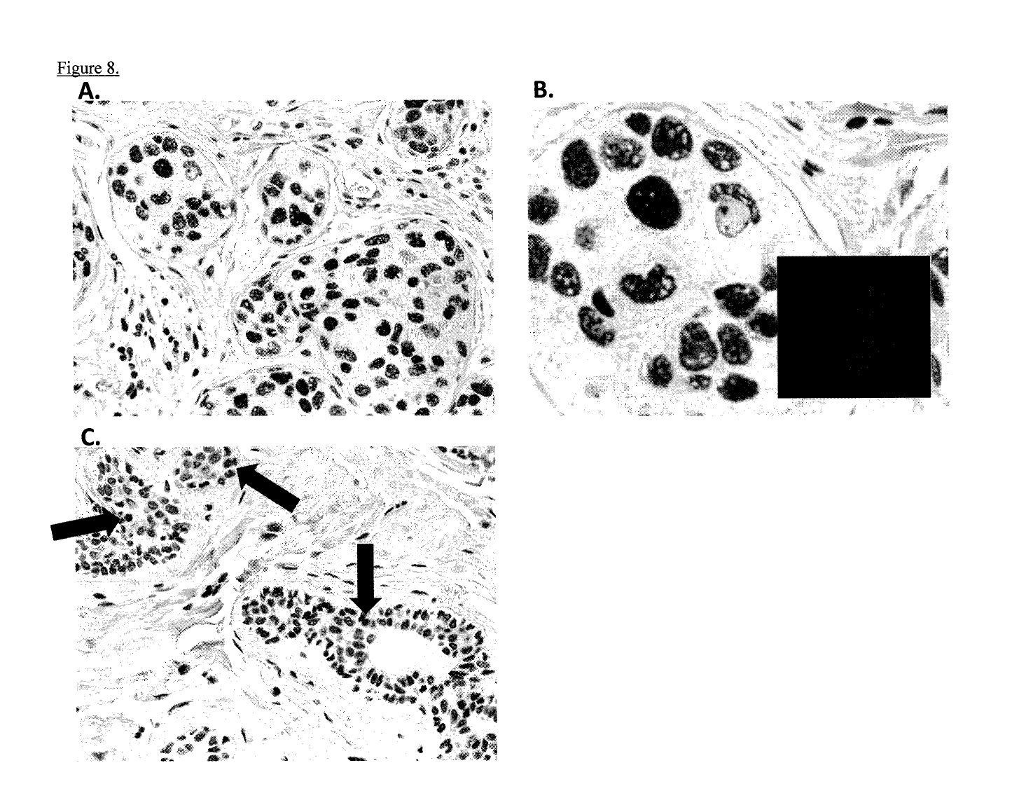

Staining for TOX3 in breast tumor tissue samples s using AJ33 demonstrates high levels of TOX3 expression in tumor, as shown by the dark, compact positive staining (FIGS. 7A, 8A and 8B). Importantly, while anti-TOX3 AJ33 antibody is capable of distinguishing adjacent normal breast tissue, demonstrating almost no positive staining for TOX3 (FIGS. 7B and 8C), some cells within adjacent normal breast tissue do exhibit light positive staining for TOX3. Thus, anti-TOX3 antibodies, such as AJ33, are capable of specific identifying tumor cells, and also, potential sites within normal tissue from which tumorigenic cells may originate. These locations of TOX3 expression in normal tissues may provide important clues for understanding the genesis and character of molecular sub-types for different breast cancers.

### Example 8

### Animal Model for TOX3 Expression

Characterization of in vitro TOX3 gene and protein expression levels is complimented by in vivo studies using animal models. As such, the inventors clarified the role of TOX3 in initiating tumorigenesis by developing a novel in vivo animal model for expression of TOX3 in breast tissue. The above examples focus on a continuing role for TOX3 in tumor maintenance or progression. However, TOX3 may be an initiator of disease rather than maintenance of the tumor phenotype, as TOX itself plays a transient role during development of the immune system (FIG. 1). In this context, creation of an in vivo model system is particularly important to allow mechanistic dissection of the role of TOX3 in breast cancer, including tumor induction. Data disclosed herein suggest that overexpression rather than mutation or loss of TOX3 likely is involved in disease; thus a conditional deletion or mutation of the protein in vivo is generated.

To determine whether alterations in TOX3 expression can directly induce cancerous changes in breast tissue or increase susceptibility to cancer, transgenic mice were produced that highly express this nuclear factor specifically in the breast. In addition, mice were generated with reversible transgene expression, such as with a tetracycline-inducible system, which gives finer control of timing of expression and allow experiments to distinguish a role for the protein in induction versus maintenance of tumors. To accomplish this, human TOX3 has been cloned from a highly-expressing tumor sample (FIG. 3), by high fidelity RT-PCR. For the reasons presented above, primers were designed to clone TOX3 variant 1 for this purpose. From preliminary sequence analysis (based on independent PCR reactions) a single silent polymorphism in the coding region from this patient's tumor, when compared to the public database sequence, is found. (FIG. 5A) This is consistent with the inventors' belief that mutations in the coding sequence of TOX3 are not associated with breast cancer, while level of expression is. This most basic issue has not been addressed in the context of breast cancer.

Transgenic mice were produced using a mouse mammary tumor virus (MMTV) promoter-based expression vector obtained from Dr. Windle (Virginia Commonwealth University). The human TOX3 cDNA was inserted into exon 3 of the rabbit beta globin gene in this vector. There are no translation start sites in the globin sequences upstream of the cDNA, but there is an upstream exon/intron to allow splicing, necessary to obtain expression in transgenic mice. This vector gives high-level expression in breast tissue in vivo (33). Genetic background can play an important role in rodent tumor models, as it does in human disease. These transgenic mice were produced in the FVB/N strain, an easy strain for production of transgenic mice, but most importantly, also found to be susceptible to mammary tumor formation by expression of various genes under control of the MMTV promoter, including ErbB2 (Her2/neu) (as described herein), Hrasl (34), and Wnt1 (35).

First generation progeny of transgenic founder mice (MMTV-huTOX3 Tg) are screened for expression of TOX3 in breast tissue by RT-PCR, and via Western blot using the antibody produced as described herein. The rabbit β-globin untranslated sequence allows specific detection of the transgene, both at the level of RNA and DNA by PCR. Strains with high level of expression are bred for additional characterization. Two types of analyses are conducted. First, spontaneous tumor formation in these mice is examined. Second, the ability of TOX3 expression to modulate oncogene-driven tumor formation as described below is analyzed. These experiments are conducted simultaneously, as the former also acts as a control for the latter.

Mammary glands from MMTV-huTOX3 Tg and wildtype littermate virgin mice were examined at 5 weeks, 2 months, and 4 months postpartum to look for structural differences, and of course tumor formation. Since, TOX3 appears to be associated with ER+ disease it is also possible that there may be effects induced by hormone responsiveness. To test this, MMTV-huTOX3 Tg and wild type littermate mice during pregnancy are compared. The mouse mammary gland undergoes well-characterized differentiation changes during pregnancy and lactation that might affect TOX3 activity (36).

The ability of TOX3 to modulate the timing, incidence, phenotype, or progression of disease induced by ErbB2 (Her2/neu) is examined. Although the ErbB2 subtype is more associated with ER− disease, preliminary data has indicted that there can be overlap in expression of ErbB2 and TOX3 in breast cancer tumors. There are two relevant transgenic mouse models, both on a FVB/N background, and both commercially available that express ErbB2 under the MMTV promoter and lead to disease. In one (37), expression of unactivated rat ErbB2 in mice leads to focal mammary tumors that first appear at 4 months. There is also a high frequency of secondary metastatic disease in the lung. In the other model (38), expression of a transforming mutated version of rat ErbB2 results in multifocal disease involving the whole epithelium.

These mice are utilized to determine the level of up regulation of endogenous TOX3 in ErbB2-induced tumors. These induction experiments allow comparison between the incidence of expression of TOX3 in focal and multi-focal disease. In addition, these Tg lines are bred to MMTV-huTOX3 Tg produced as described herein, to determine if disease induction or progression is affected. Given the relatively long lag time for disease induction in ErbB2 Tg mice, TOX3 expression may supply a “second hit” to promote disease, thereby causing a significant shift in kinetics. Development of an inducible animal model system allows observation of these in vivo changes, that result from expression of TOX3.

### Example 9

TOX3 sequences

TOX high mobility group box family member 3 [Homo sapiens]
(SEQ ID NO: 1):
MDVRFYPAAAGDPASLDFAQCLGYYGYSKFGNNNNYMNMAEANNAFFAASEQTFHTP
SLGDEEFEIPPITPPPESDPALGMPDVLLPFQALSDPLPSQGSEFTPQFPPQSLDLPSITISRN
LVEQDGVLHSSGLHMDQSHTQVSQYRQDPSLIMRSIVHMTDAARSGVMPPAQLTTINQ
SQLSAQLGLNLGGASMPHTSPSPPASKSATPSPSSSINEEDADEANRAIGEKRAAPDSGK
KPKTPKKKKKKDPNEPQKPVSAYALFFRDTQAAIKGQNPNATFGEVSKIVASMWDSLG
EEQKQVYKRKTEAAKKEYLKALAAYRASLVSKAAAESAEAQTIRSVQQTLASTNLTSSL
LLNTPLSQHGTVSASPQTLQQSLPRSIAPKPLTMRLPMNQIVTSVTIAANMPSNIGAPLISS
MGTTMVGSAPSTQVSPSVQTQQHQMQLQQQQQQQQQQMQQMQQQQLQQHQMHQQI
QQQMQQQHFQHHMQQHLQQQQQHLQQQINQQQLQQQLQQRLQLQQLQHMQHQSQP
SPRQHSPVASQITSPIPAIGSPQPASQQHQSQIQSQTQTQVLSQAIPTICESNCLMNPGTY
N-terminus of TOX high mobility group box family member 3
[Homo sapiens] (SEQ ID NO: 2):
MDVRFYPAAAGDPASLDFAQCLGYYGYSKFGNNNNYMNMAEANNAFFAASEQTFHTP
SLGDEEFEIPPITPPPESDPALGMPDVLLPFQALSDPLPSQGSEFTPQFPPQSLDLPSITISRN
LVEQDGVLHSSGLHMDQSHTQVSQYRQDPSLIMRSIVHMTDAARSGVMPPAQLTTINQ
SQLSAQLGLNLGGASMPHTSPSPPASKSATPSPSS SINEEDADEANRAIGEKRAAPDSG
TOX high mobility group box family member 3 DNA [Rattus norvegicus]
(SEQ ID NO: 3):
atggatgtga ggttctaccc cgcggcggcc ggggatcccg ccggcctgga cttcgcgcag
tgcctggggt actacggcta cagcaagttg ggaaataata actacatgaa catggctgag
gcaaacaacg ccttctttgc tgccagtgag cagacattcc acacgccaag ccttggggat
gaagagtttg aaattccgcc gatcacgcct cctccagagt cagaccccac cctgggcatg
cccgatgtac tgctaccctt tcagacactc agcgatccgt tgccttccca gggaaatgag
ttcacacccc agtttccccc tcagagcctg gatcttcctt ccatcacaat ctcaaggaat
ctggtggagc aagatggtgt gcttcatagc aacgggctgc atatggatca gagccacaca
caagtgtcgc agtaccgcca ggatccttct ttggtcatga ggtcaattgt ccacatgaca
gatgctgctc gctctgggat catgcctcct gcccaactga ccaccatcaa ccagtctcag
ctcagtgcac agttgggctt gaatctggga ggagccagtg tgccccacac gtctccttca
cctccagcaa gcaaatcagc cactccctcc ccttccagct ctatcaatga agaggatgct
gatgaaacaa acagagccgt tggagagaaa agaacagccc cagattctgg caagaagccc
aagactccaa agaaaaagaa aaagaaagat cccaacgagc ctcagaagcc agtgtcagca
tacgccctgt ttttcagaga tacacaggct gcaattaaag gtcaaaaccc caatgcgacc
tttggagaag tctcaaaaat tgtagcatct atgtgggaca gccttggaga ggagcaaaag
caggtatata aaaggaaaac agaagctgcc aagaaagaat atttgaaggc cctggctgcc
taccgggcca gcctcgtttc taaggctgct gctgagtccg cagaagccca gactatccgc
tctgtccagc agactctggc atcaaccaac ctgacatcct ctctccttct gaacacgtca
ctgtctcaac atgggacagt cccggcttca cctcagactc tcccgcagtc actccctagg
tcgattgccc ccaaaccctt aaccatgaga ctacccatga gccagattgt cacatcagtc
accattgcag ccaacatgcc ctcgaacatt ggggctccac ttatcagctc catgggaacg
accatggttg gctcagtatc ctccacgcag gtgagccctt cggtacaaac ccagcaacat
cagctgcagc tgcagcagca gcaacaacag cagcagcagc agatgcagca gatgcaacat
cagcagctgc agcagcacca gatgcatcag cagattcagc agcagatgca gcagcagcat
ttccagcacc acatgcaaca gcacctgcag cagcagcaac agcagcacct ccagcagcag
atcagccaac agcagctgca gcagcagctg cagcagcatc tccagctgca gcagcagctg
cagcacatgc agcaccagtc tcagccttct ccccggcagc actcgcccgt cacctcacag
atcacgtccc ccatccccgc cattggcagc ccccagccag cctctcagca gcaccagcct
caaatccagt cgcagacaca gactcaagtg ttaccgcagg tcagtatttt ttaa
TOX high mobility group box family member 3 DNA (trinucleotide
repeat containing 9, transcript variant 1 (TNRC9)) [Homo sapiens]
(SEQ ID NO: 4):
gcggccgcgg ctcccgagct cctcgggctc tgggtcccgg cgcccctccg gccgcgagtc
ccacgcgcca cccccgggcg ccctcgacgg tggatctagc ggcggcgagg aggcgggtcc
cggccccggc gaaccccagt cccggccccc ggccccgggc ccagcttcgg catggatgtg
aggttctacc ccgcggcggc cggggaccct gccagcctgg acttcgcgca gtgcctgggg
tactacggct acagcaagtt tggaaataat aataactata tgaatatggc tgaggcgaac
aatgcgttct tcgctgccag tgagcagaca ttccacacac caagccttgg ggacgaggaa
ttcgaaattc caccaatcac gcctcctcca gagtcagacc ctgccctagg catgccggat
gtactgctac cctttcaagc cctcagcgat ccattgcctt cccagggaag tgaattcaca
ccccagtttc cccctcaaag cctggacctc ccttccatta caatctcaag aaatctcgtg
gaacaagatg gcgtgcttca tagcagtggg ttgcatatgg atcagagcca cacacaagtg
tcccagtacc ggcaggatcc ctccctgatc atgcggtcca tcgtccacat gaccgatgct
gcgcgttctg gggtcatgcc tcctgcccag ctcaccacca tcaaccagtc tcagctcagc
gcccagttgg ggttgaattt gggaggtgcc agtatgcctc acacatctcc ttcacctcca
gcaagcaaat cagccactcc ctccccttcc agctccatca atgaagagga tgctgatgaa
gccaacagag ccattggaga gaaaagagct gctccagact ctggcaagaa gcccaagact
ccaaagaaaa agaaaaagaa agatcccaat gagccacaga agccagtgtc agcatatgcc
ctgtttttca gagacacaca ggctgcaatt aaaggtcaaa accccaatgc aacctttgga
gaggtctcaa aaattgtagc atctatgtgg gacagccttg gagaagaaca aaagcaggta
tataaaagga aaacagaagc tgccaaaaaa gaatacctga aggccctggc ggcatacagg
gccagcctcg tttctaaggc tgctgctgag tcagcagaag cccagaccat ccgttctgtt
cagcagaccc tggcgtcgac caatctaaca tcctctctcc ttctcaacac tccactgtct
caacatggaa cagtgtcagc atcacctcag actctccagc aatccctccc taggtcaatc
gctcccaaac ccttaaccat gagactcccc atgaaccaga ttgtcacatc agtcaccatt
gcagccaaca tgccctcgaa cattggggct ccactgataa gctccatggg aacgaccatg
gttggctcag caccctccac ccaagtgagt ccttcggtgc aaacccagca gcatcagatg
caattgcagc agcagcagca gcagcaacaa caacagatgc aacagatgca gcagcagcaa
ctccagcagc accaaatgca tcagcaaatc cagcagcaga tgcagcagca gcatttccag
caccacatgc agcagcacct gcagcagcag cagcagcatc tccagcagca aattaatcaa
cagcagctgc agcagcagct gcagcagcgc ctccagctgc agcagctgca acacatgcag
caccagtctc agccttctcc tcggcagcac tcccctgtcg cctctcagat aacatccccc
atccctgcca tcgggagccc ccagccagcc tctcagcagc accagtcgca aatacagtct
cagacacaga ctcaagtatt atcgcaggct atacctacaa tatgtgaatc aaactgttta
atgaatcctg ggacatactg atgactataa actggcctct ctgagtcata gaaaaatggc
cttatttctc cagaagtgag taaaccacac ttccaggcta tctgaactcc tgaagcccta
aaaataaaaa gcacagttgt aactacctga aatatgaaga tccagtttca tacaaacatt
tgtatgacgt gaatagttga tggcattttt ttgtcatgaa aaaaataatg taaatcacag
acttttgcca aagctcttat tttttttcct aaatctctcc agaaaaaaaa tgcaagtgac
taaattcaat tattgactaa tttccacttt ttatccatga cttctccaaa tcaaaccaca
gtatatgttg taacaatatc tatgaccact gttagcccat tatattcatt ccaattagaa
gaaatgtgaa tactatattc cgtgttttga gtgacaagtt tcgaaaaata aaaacactgt
atttttaaaa gggaaatgca cttaaatgaa aacagttatt acaaaagtta agatttaaaa
agaaaaagca agagttttta ttatgatgta ataccagtag aatatttaaa aggcacacca
catctgaata atcaatgtaa atattttctt tcaaagttgt aagttttcat atcatgtgct
gtaaagtttt cctaaatgag gctttaacgt aaacactggt gacataaacc attcattgct
acgttgctta ttgtgttttt atgctgtttt atactttttt atgagttatg atagcagcaa
ttaagttgtt tgtattttgc ttaactaaaa caaaaatgct tttatcttgc tatagaataa
acacatttca gtaaaaactg tggactgtat tttgatgcaa caacaaagaa actgttcact
tttcaaataa aatgatatgt cagatttca

### Example 10

### Expression of TOX3 in Mammary Epithelium Primarily Occurs in ER+Luminal Cells

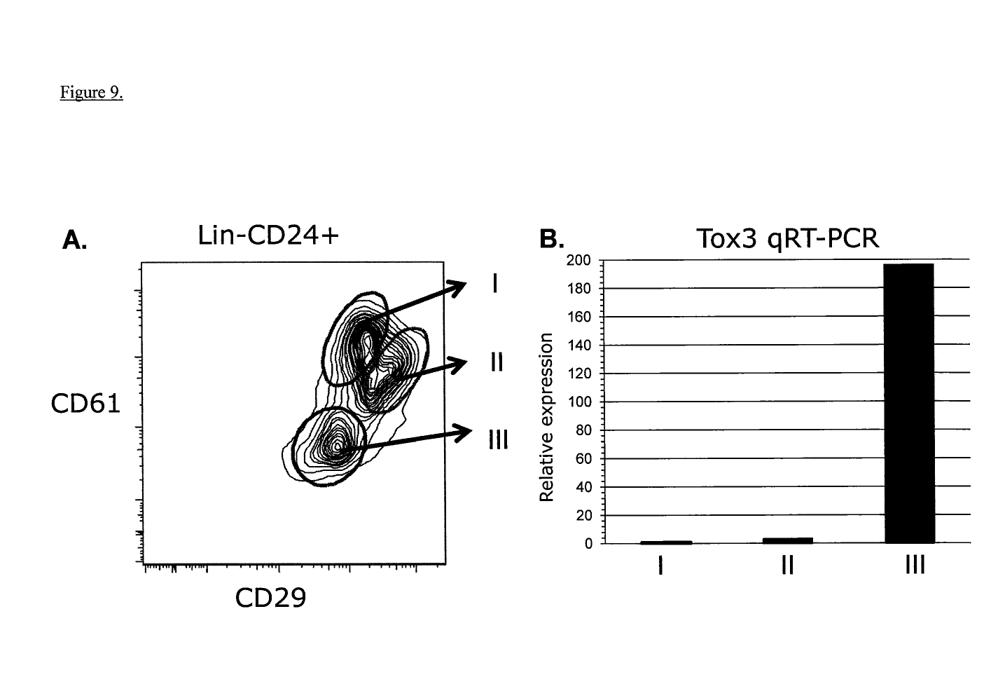

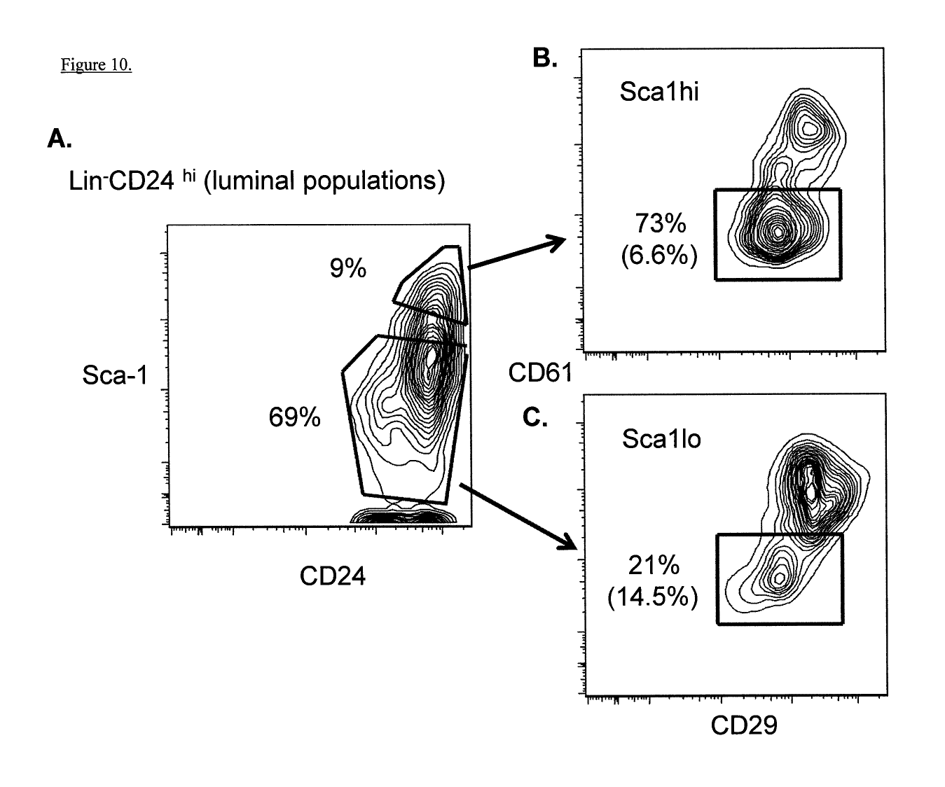

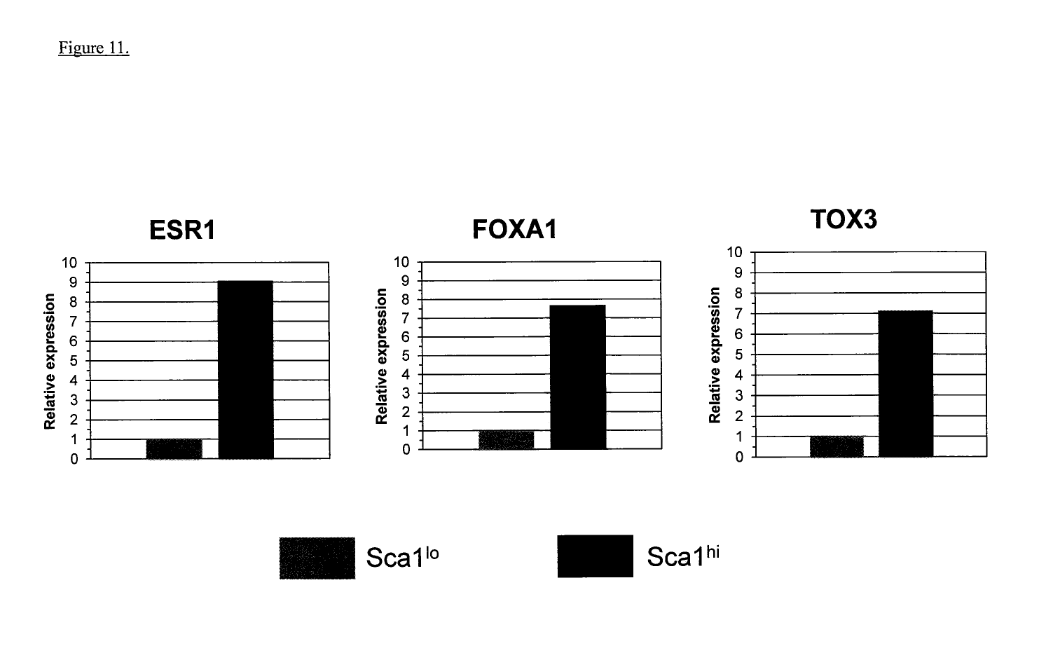

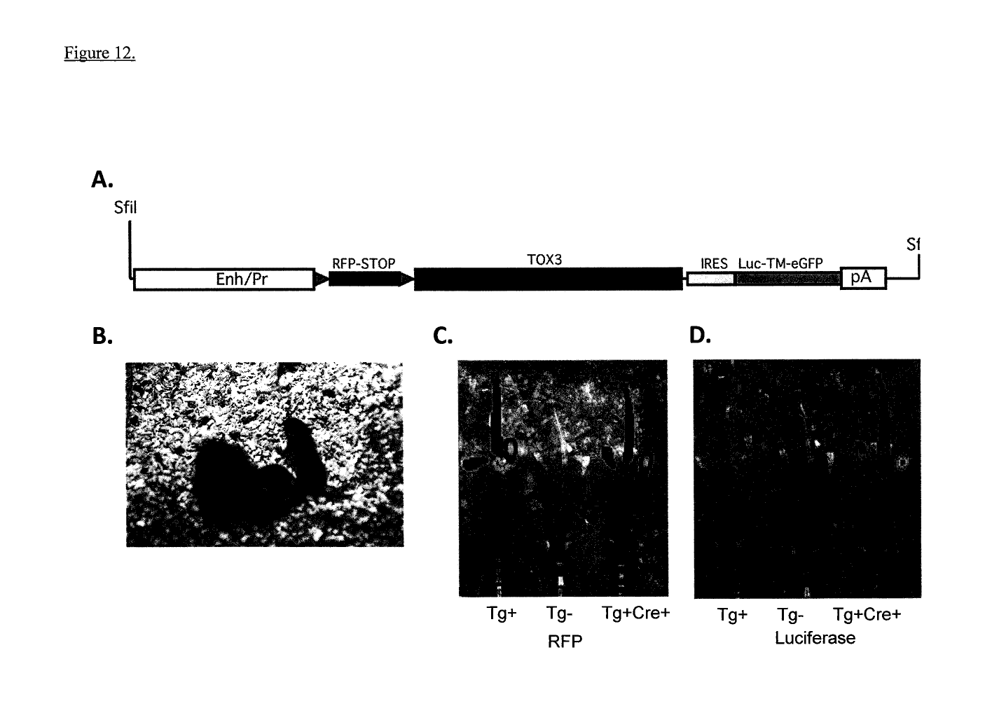

Cell sorting was performed that identified populations of precursor for luminal cells, mammary epithelial “stem” cells, and mature luminal cells (FIG. 9A). Among these three categories cells that are found in the mammary epithelium population, mature luminal cells exhibited a remarkably higher amount of TOX3 expression compared to less differentiated precursor, “stem” cells, and basal layer cells (FIG. 9B). Luminal populations were first sorted for stem cell antigen-1 (SCA-1) and CD24 expression to isolate cells that serve as precursors, progenitors, and immature cells for mammary gland development and maintenance (FIG. 9A). Comparison against populations further sorted against CD61 and CD29, indicate SCA-1^{hi} is predominantly (73%) associated with lower CD61 expression, while SCA-1^{lo} is not (21%). (FIG. 9B). Further measurement of estrogen receptor alpha (ESR1), FOXA1, and TOX3 expression in normal mammary epithelium indicates these genes are significantly upregulated in SCA-1^{hi} population (i.e., a ER⁺ subset of luminal cells) (FIG. 10). These results clearly indicate a strong association of TOX3 expression with the ER⁺ population of luminal epithelial cells, these cells forming the inner lining of organ ducts in the mammary gland. Additional studies with a cre-activated TOX3 transgenic animal confirm that TOX3 plays a significant role in mammary epithelial cell development (FIG. 12).

### Example 11

### TOX3 Expression Across Breast Cancer Subtypes

As described earlier, breast cancer can be organized into 5 major molecular subtypes (41), although more recent studies have refined this classification into 6 subtypes (after further division of a previous ErbB2⁺subtype into mApo and luminal C subtypes), based on 537 primary breast transcriptomes and 256 gene signature. These 6 subgroups differ in signaling pathways activated, propensity for metastatic relapse to brain vs. bones, therapeutic responses, and include the following: luminal B (high proliferative), luminal A (low proliferative), normal-like (low proliferative), luminal C, molecular-apocrine Apo (AR⁺), basal like (AR⁻). Luminal A, B, and normal-like breast cancers express high levels of ER, whereas Luminal C express lower levels of ER, and molecular Apo and basal-like cells are both ER and PR negative (FIG. 17B).

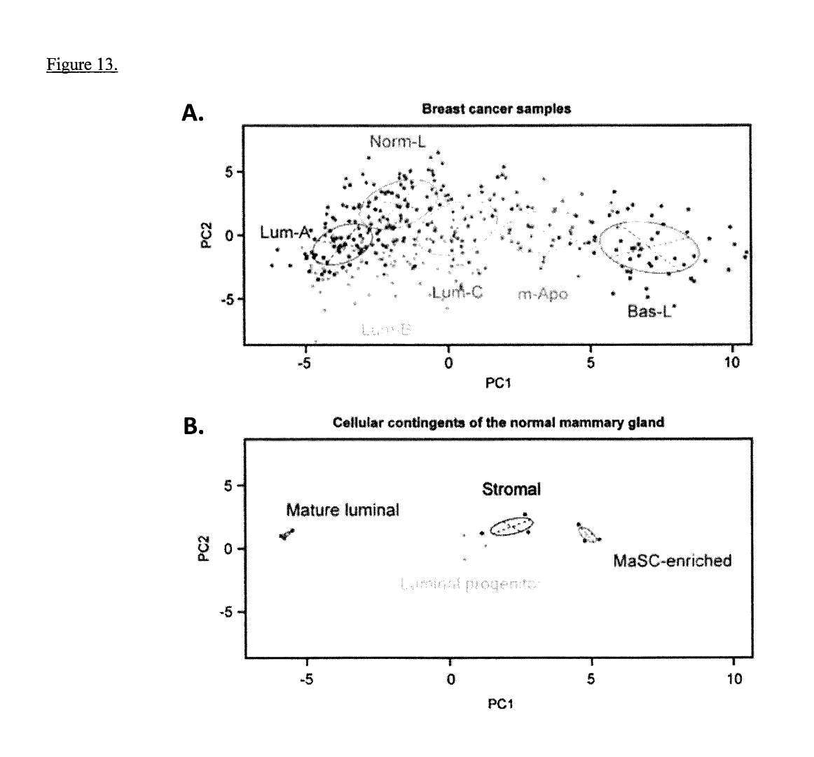

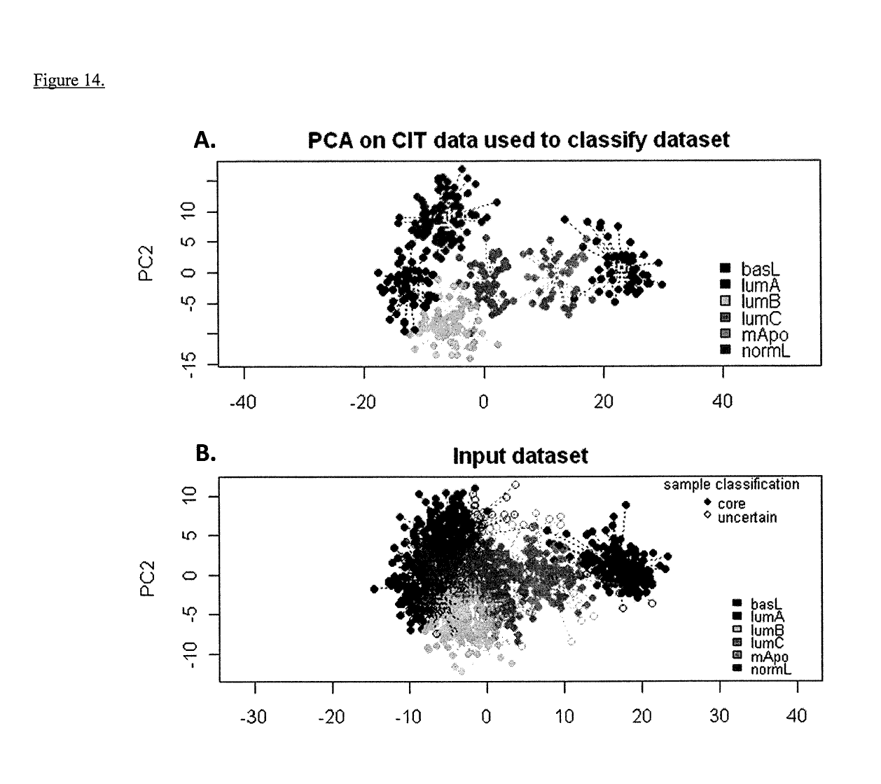

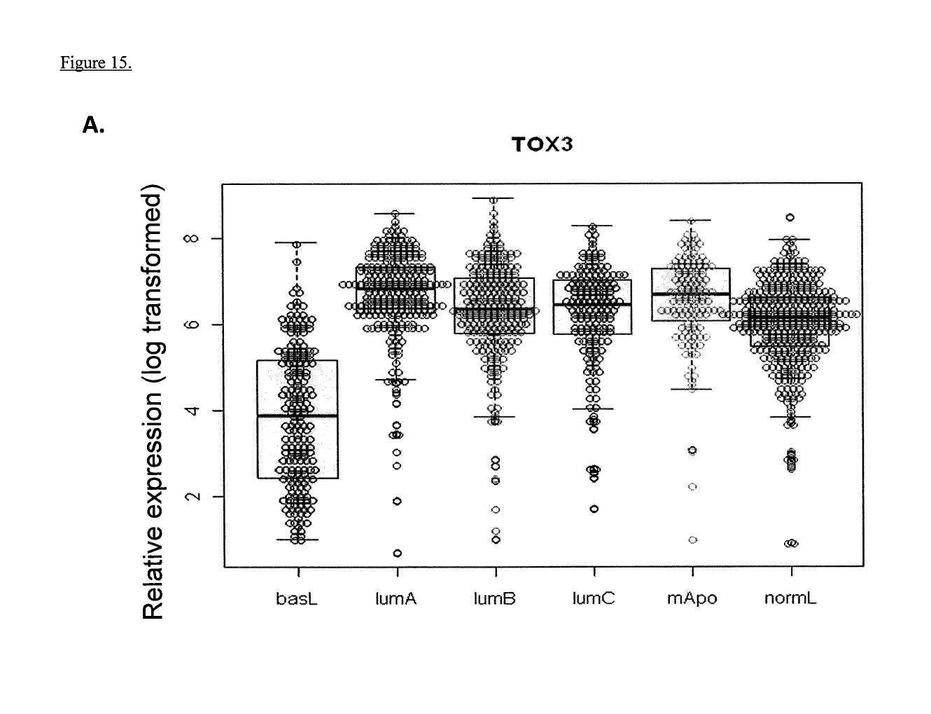

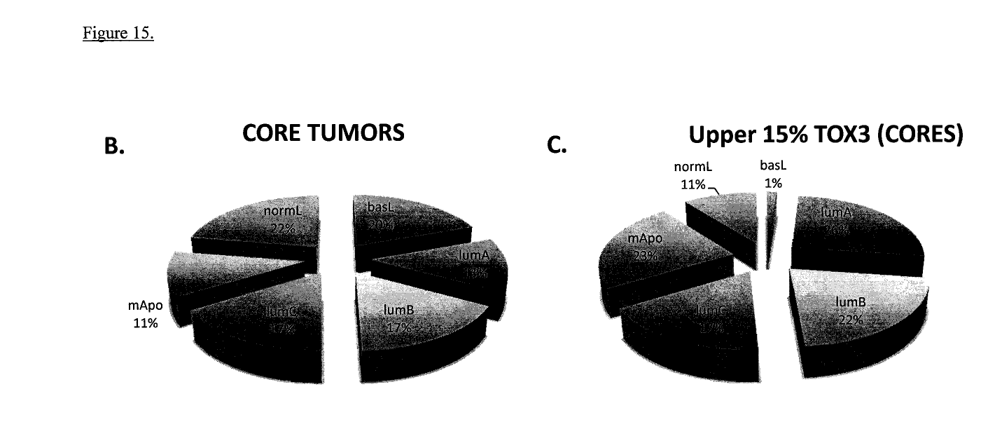

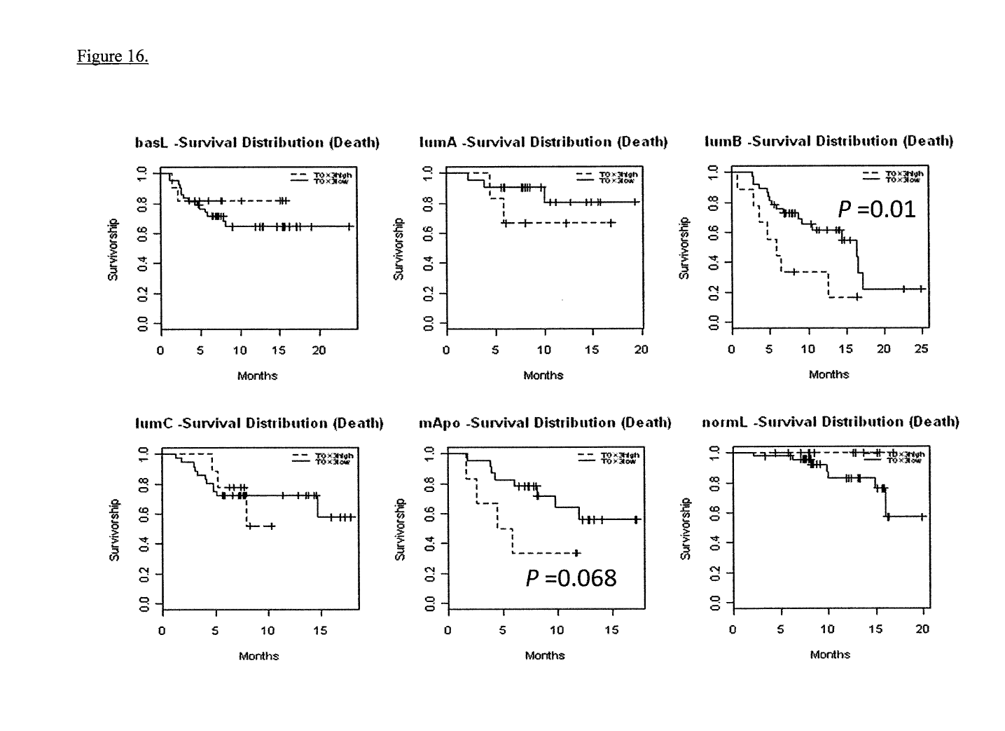

Classification of molecular subtypes of breast cancer provides a useful context for contrasting the role of TOX3, particularly because TOX3 appears to play a role in cellular morphogenesis and differentiation. In particular, principal component analysis of this molecular heterogeneity of breast cancer indicates a relation to cell of origin and/or differentiation state of tumor. For example, luminal A cancer may arise from mature luminal cells, whereas basal-like cancers may originate from MaSC-enriched populations (FIGS. 13A and 13B). Applying these categorizations to publicly available microarray data for 1391 breast cancer tumors further confirms the molecular heterogeneity of breast cancer along approximately six subytpes. The vast majority, 957 tumors, indicated a “core” phenotype of one sub-type category, with 375 tumors being “outliers” along a subtype, and only 60 displaying a “mixed” molecular signature (FIGS. 14A and 14B). TOX3 is highly expressed in all tumor subtypes compared to healthy tissue (FIG. 15). While TOX3 expression can be found across nearly all breast cancer subtypes (FIGS. 15A and 15B), evaluating only the upper 15% threshold of TOX3 expression provides the surprising result that highest TOX3 expression is mostly found in luminal and mol. Apo breast cancer tumors (ranging from 17-26%), and fewer population members that are basal-like cells (11%) and virtually non-existent in normal-like cells (1%) (FIG. 15C). This affirms the view that levels of TOX3 expression, rather than mutation or deletion, is a key property in the genesis of a particular molecular subtype of breast cancer.

### Example 12

### Clinical Features of TOX3 Expression

Interestingly, TOX3 expression is associated not only with particular breast cancer molecular subtypes, but this association is further correlated with a shorter time-to-death. For example, high TOX3 expression resulted in a statistically significant, decrease in time-to-death in mol. Apo. And luminal B cancer sub-types (FIG. 16). Similar observations, in luminal A and C sub-types were observed, although such results were not statistically significant (FIG. 16). Notably, this trend was not observed in the two subtypes, wherein high TOX3 expression occurs less frequently, basal-like and normal-like cancer subtypes.

Further, tissue array studies of 188 patients displaying invasive ductal carcinoma further suggests a key role for TOX3 in breast cancer pathogenesis. Distribution across patients for key pathological markers, ER and Her was as follows: 32% ER⁺Her2⁺, 26% ER⁺Her2⁻, 29% ER⁻ Her2⁺, and 13% ER⁻Her2⁻. Among these patient populations high TOX3 expression (>10%++), was observed in ˜16% of tumors, including 25% of ER⁺Her2⁺samples (often associated with a luminal C subtype), and 15% of ER⁻Her2⁺samples (often associated with a mol. Apo. subtype) (FIG. 17A). Other population displayed less TOX3 expression, including only 8% of ER⁻Her2⁻samples (often associated with a basal-like subtype) and 11% of ER⁺Her2⁻ (often associated with luminal A, luminal B, and normal-like subtypes) expressing high TOX3 expression (FIG. 17B). Together, the time-to-death results and tissue array studies provide a consistent picture of high TOX3 expression being associated with mol. Apo., moderate association with luminal A, B and C subtypes, and weaker association with the basal-like and normal-like subtype.

### Example 13

### TOX3 In Vitro Expression

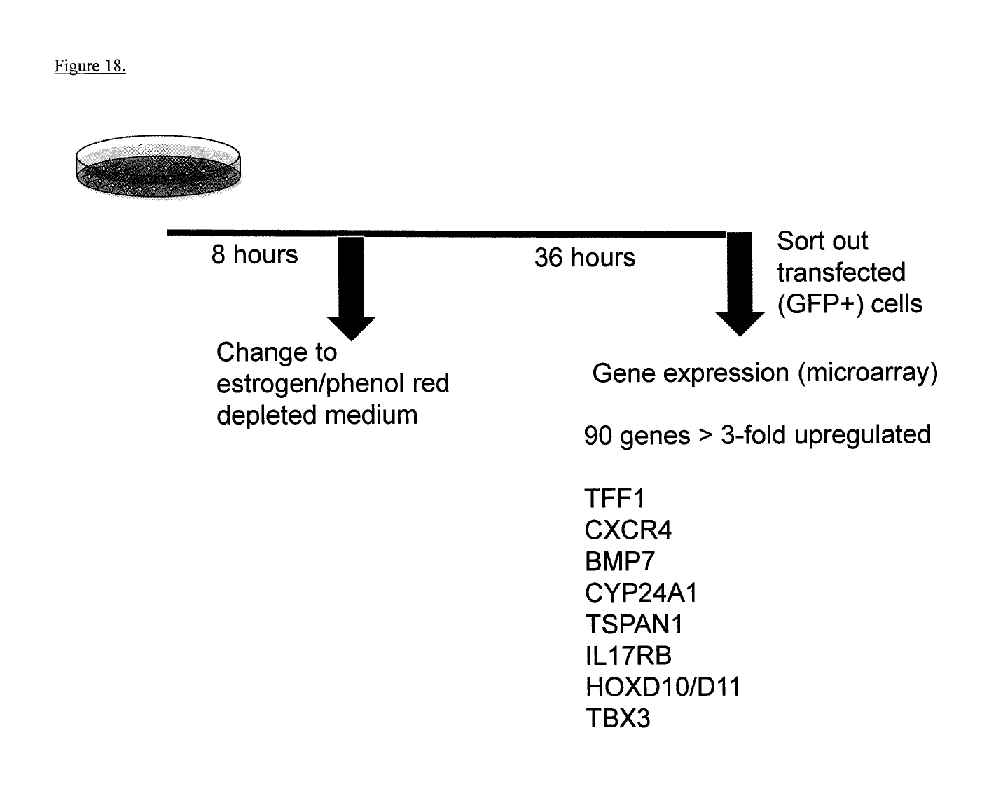

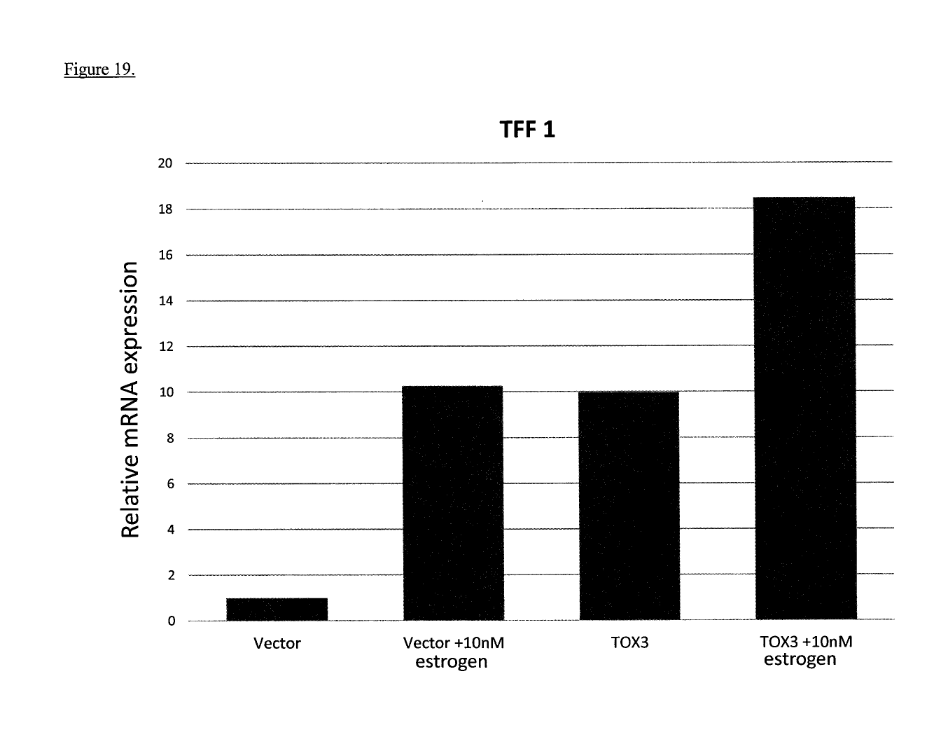

Having established that TOX3 is expressed in mammary epithelium and is associated with clinical features of breast cancer, in vitro transfection of TOX3 into breast cancer cell line, MCF-7, provides further insights into the mechanisms of TOX3 activity. Of particular interest, is an assessment the downstream expression of genes associated with potential TOX3 induction of gene expression. General study design is shown (FIG. 18). Briefly, it was observed that approximately 90 genes were upregulated by over 3-fold, 36 hours after estrogen depletion. This includes TFF1, CXCR4, BMP7, CYP24A1, TSPAN1, IL17RB, HOXD10/D11, and TBX3.

Of these genes, TFF1 and CXCR4 are of particular interest. Trefoil factor 1 (TFF1), is a well-studied gene, known to be expressed in ER+ luminal cells, also previously identified in GWAS studies (10,11) to be highly enriched in breast cancer samples. However, the role of TFF as a pro-tumorigenic oncogene, or oncogene suppressor is not entirely clear (44, 45). In breast cancer, it appears that TFF1 promotes motility, which could account for migratory cell invasion. Further, CXCR4 is a receptor well-known to be involved in stem cell migratory function. Further investigation of TFF1 and CXCR4 may suggest avenues underlying TOX3-mediated cancer pathogenesis.

### Example 14

### TOX3-Mediated Induction of TFF1

As described, TFF-1 is a well-studied estrogen inducible gene expressed in ER+ luminal cells. This protein encodes a protease resistant secretory protein also expressed in gastrointestinal mucosa. It is likely that TFF1 protects the mucosa from insults, stabilizes the mucus layer, and affects healing of the epithelium. It has been reported that TFF is a tumor suppressor in the stomach (44) and perhaps in the mammary gland (47). However, it has also been reported that TFF1 increases motility of breast cancer cell lines (45).

The results of the microarray studies were validated, as TOX3-mediated induction of TFF1 is recapitulated in stable MCF-7 cell transfectants (FIG. 19). Estrogen depletion for 48 hours, followed by estrogen treatment for 24 hours resulted in a significant increase (at least 9 fold) in TOX3 expression compared to vector control (FIG. 19).

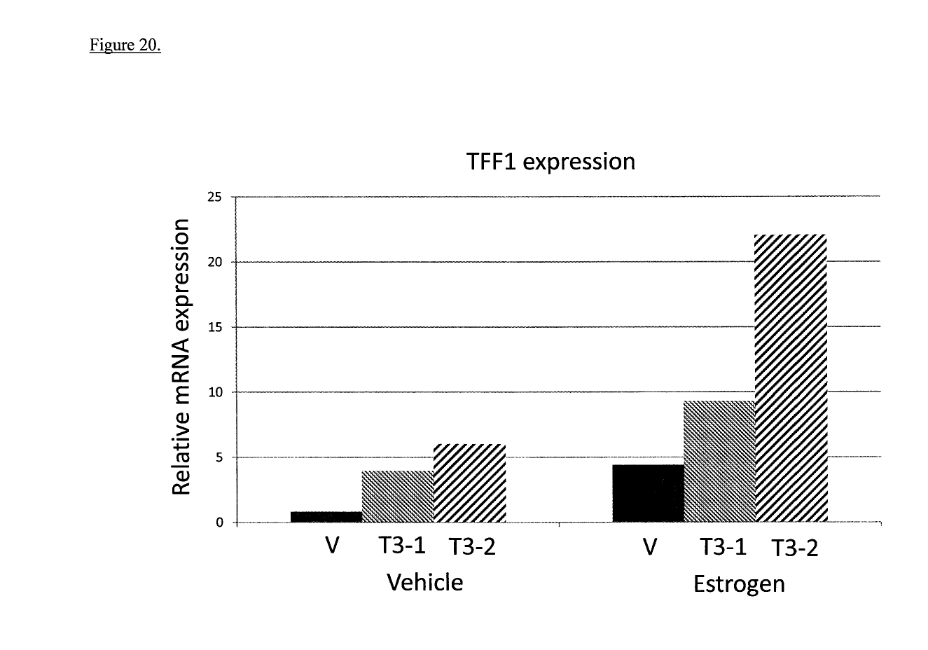

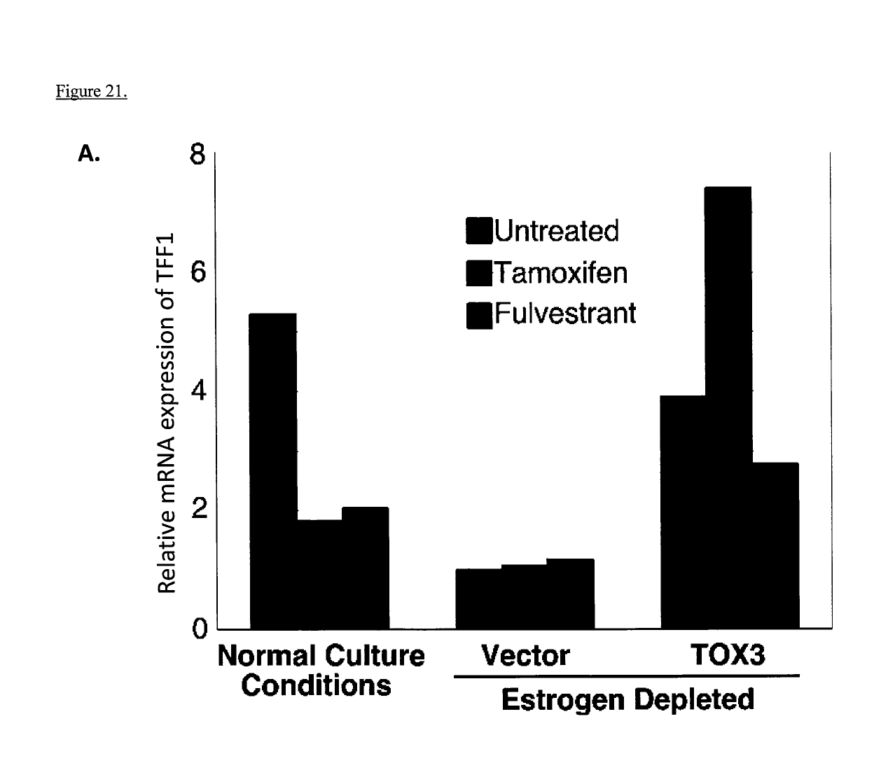

Interestingly, TOX3-mediated induction of TFF1 expression is tamoxifen resistant, but fulvestant sensitive (FIG. 20). Tamoxifen functions through two primary mechanisms: 1) blocking estrogen binding to estrogen receptor and 2) co-repression of estrogen receptor target genes. Fulvestant also blocks estrogen binding to estrogen receptor, but instead of co-repression of ER target genes at an intracellular level, fulvestant inhibits nuclear localization and promotes ER degradation. As TOX3-mediated induction of TFF1 expression is tamoxifen resistant, but fulvestant sensitive, this suggests that the mechanism of TOX3 induction operates through an unliganded ER activation.

Classically, estrogen exerts biological function via two nuclear hormone receptors, ERa and ERβ, these two receptors functioning as ligand-dependent transcription factors. Following ligand binding, ERα and ERβ, together with a number of cofactor proteins, rapidly associate with specific genomic targets. Central to this the initiation of this process is binding of a ligand, typically estrogen. Binding activation by the ligand results in phosphorylation of different sites located within the ER sequence, including several serine residues, such as serine 118 (FIG. 22). Importantly, the inventors have discovered that even under estrogen depletion conditions, residual phosphorylation occurs in both vector and TOX3 transfected cells, although this amount is substantially reduced from normal grown conditions (FIG. 22). This suggests the possibility that other ligands that are not estrogen E2, may play a role in the activation of ERs. It is of interest to understand the role of estrogen E2 independent pathways for ER activation, and to identify what roles TOX3 may have in this process. In one aspect, identifying estrogen E2 independent pathways provides important therapeutic alternatives in the case of Tamoxifen resistance, as blocking estrogen E2 activation of ER may be of limited clinical utility when resistance appears, or in those molecular subtypes wherein E2 activation is not a dominant mechanism for cancer pathogenesis.

### Example 15

### TOX3 Influences Unliganded ER Activation

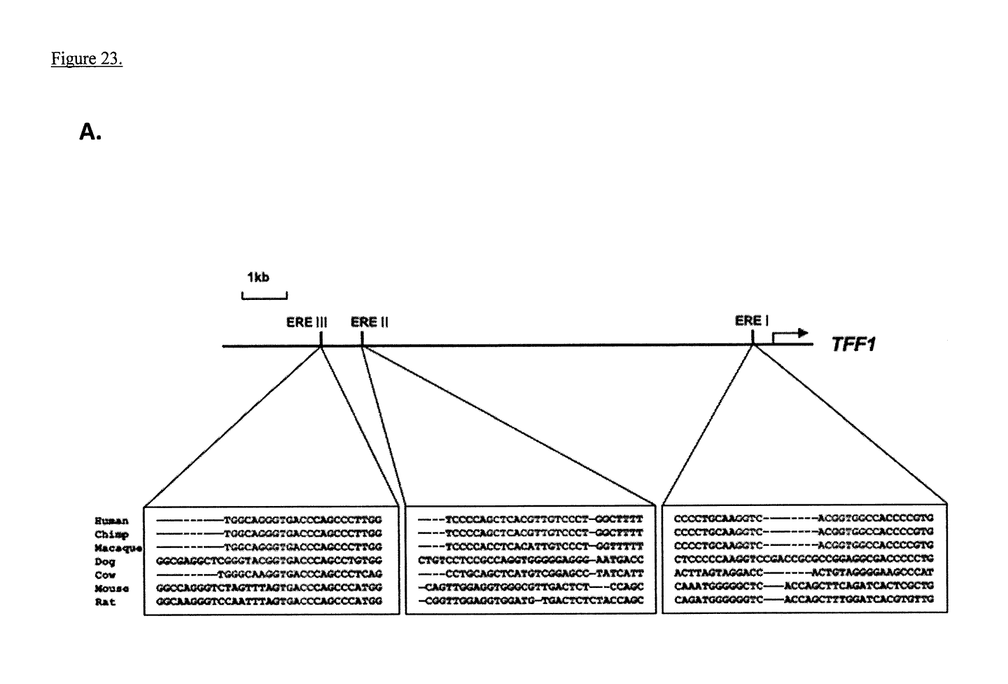

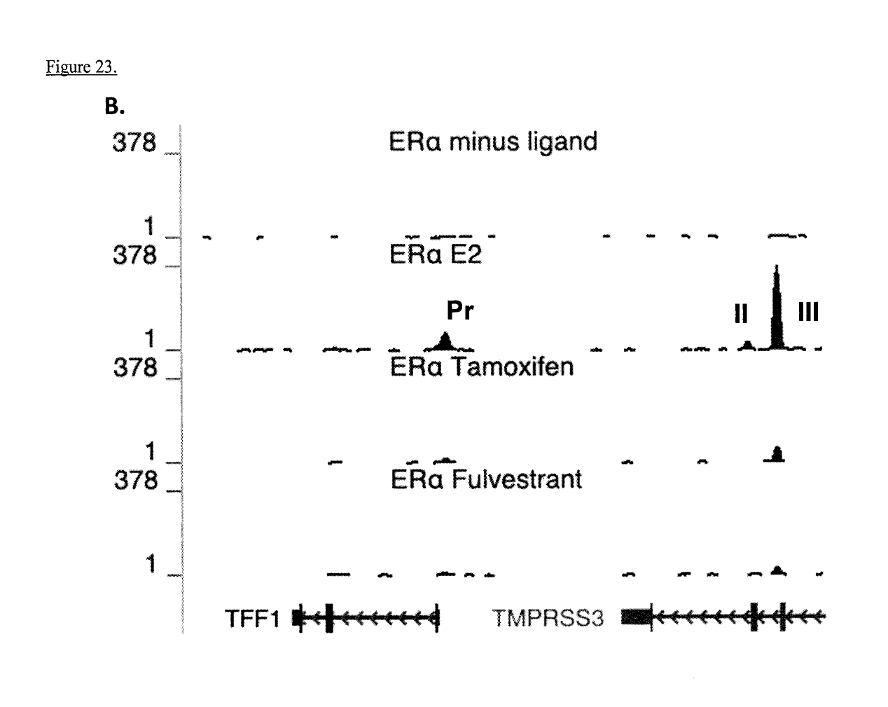

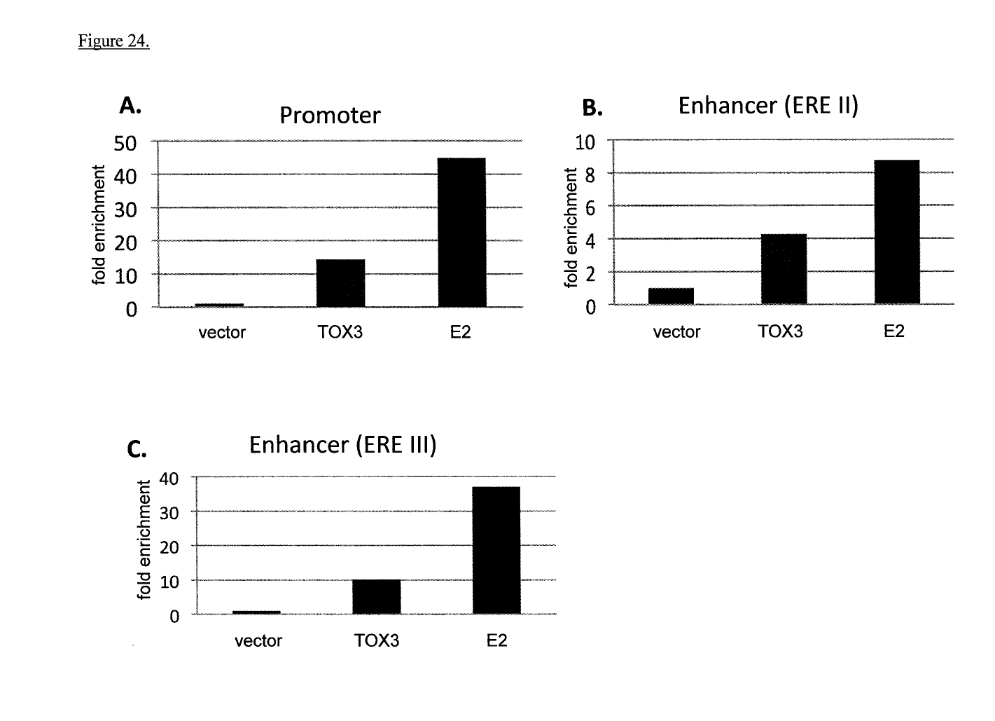

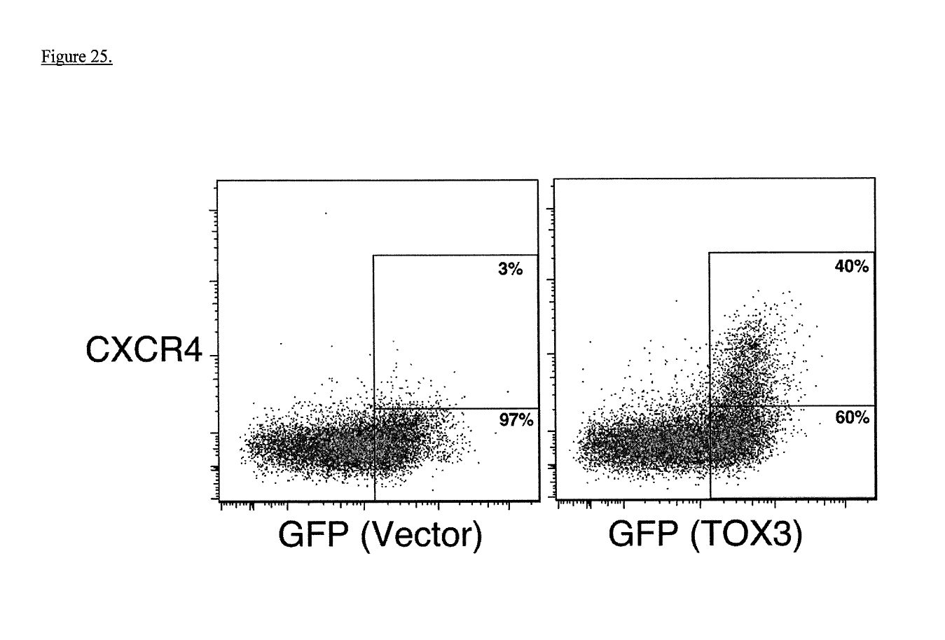

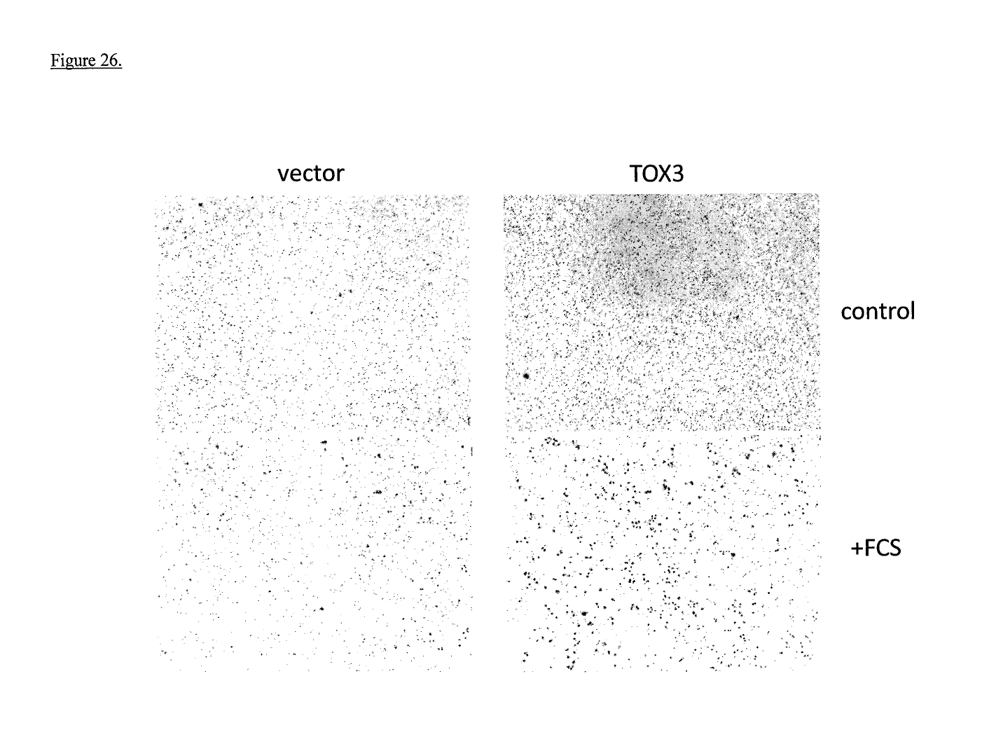

A novel mechanism for unliganded ER activation is supported by reported studies of ER CHIP studies on the promoter regions of TFF1 and related gene, TMPRSS3, both genes located in the surrounding flanks of the ERE I (i.e., promoter), II, and III sites of ER binding (46) (FIG. 23A). In these ER ChIP studies, estrogen E2 stimulation clearly results in a binding to ERE I, II, and III binding sites. However, even after application of tamoxifen, fulvestrant, or in the absence of ligand binding, residual binding is still observed at these sites found upstream of the TFF1 and TMPRSS3 start sites (FIG. 23B).

Similar results focusing on TOX3 are confirmed by the inventors' own studies in TOX3 expressing MCF-7 stable transfectants. ER ChIP studies at the TFF1 locus, demonstrated significant fold enrichment of both TOX3 and estrogen E2 at ERE I, II, and III sites compared to vector control (FIG. 24). These results shown that even under estrogen depleted conditions, TOX3 expressing cells contain significantly higher level of ER binding at ERE I, II, and III sites. (12×, 4×, and 10× higher at ERE I, II, and III sites compared to control, respectively). Estrogen E2 stimulation was higher compared to unstimulated TOX3 expressing cells (3.5×, 2×, and 3.7× higher at ERE I, II, and III sites compared to control, respectively).

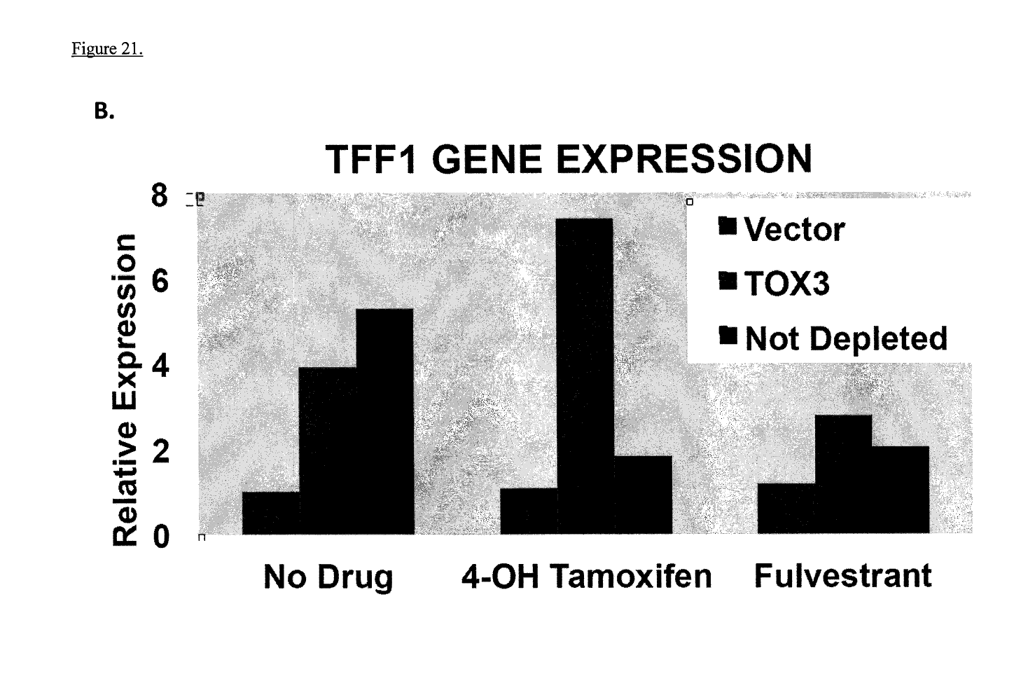

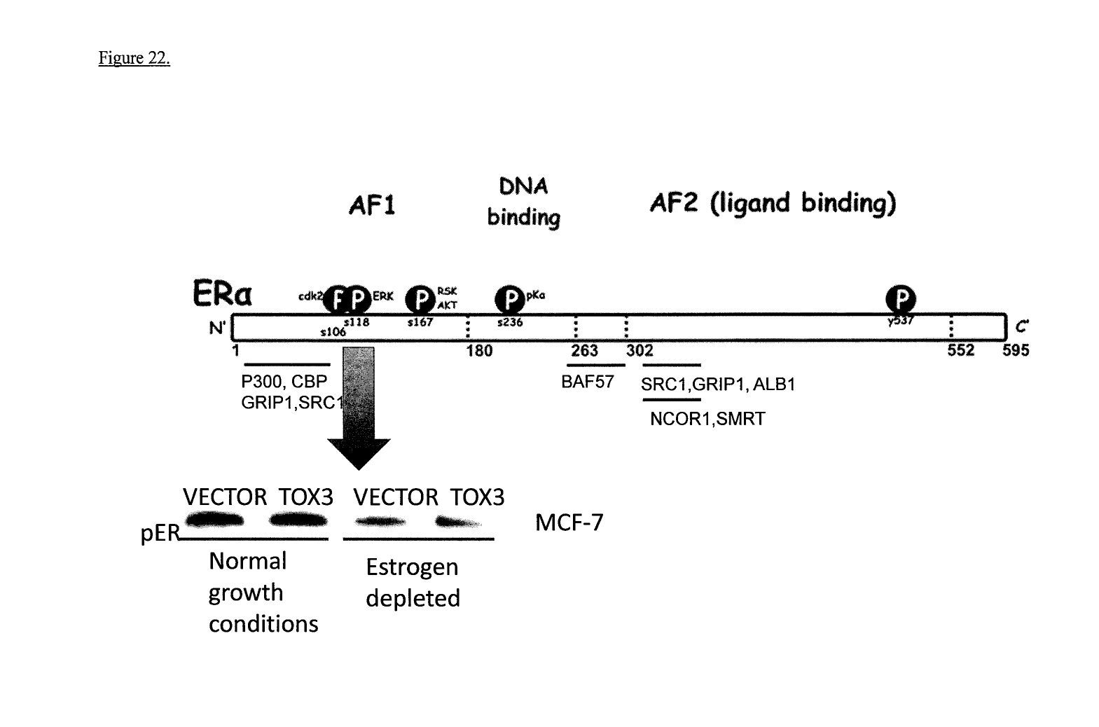

These remarkable results support a model, wherein TOX3 is able to upregulate ER-target gene TFF1, in the absence of estrogen, and even following application of tamoxifen. While residual phosphorylation of ER occurs even under estrogen depleted conditions in vector and TOX3 transfected cells like (FIG. 22), it is clear that TOX3 expressing cells exhibit enhanced ER binding to ERE sites compared to vector control and in the absence of estrogen E2 stimulation (FIG. 24). This suggests ER activation occurs independent of estrogen E2 binding, a result further affirmed by tamoxifen resistance, but fulvestant sensitivity (FIG. 21). This is because the tamoxifen antagonism of estrogen E2 binding to ER would have little or no effect in an estrogen E2 independent mechanism of TOX3-mediated TFF1 induction. Fulvestant would remain effective in halting TOX3-mediated TFF1 induction by limiting nuclear localization of ER and promoting ER degradation. By contrast, Tamoxifen's mechanism of preventing estrogen E2 binding to ER would not have an effect on a TOX-3 induction mechanism, which is independent of estrogen E2 activation of the ER. Together, these results support not only a novel observation of TOX3-mediated induction of TFF1 expression, but also a novel mechanism of unliganded ER target gene activation. As Tamoxifen resistance is a clinical result observed in many breast cancer patients, identification of this key TOX3 mediated pathway allows targeting of molecular subtypes wherein Tamoxifen delivers less effective treatment, and alternative approaches for applying therapeutic compounds after Tamoxifen resistance has emerged.

### Example 16

### TOX3 Induces CXCR4 Expression

Further studies in TOX3 MCF-7 stable transfectants demonstrate enriched expression of CXCR4 compared to vector controls. Using cell sorting against CXCR4 and GFP labels, only 3% of vector control cells expressed CXCR4, whereas 40% of TOX3 stable transfectants expressed this marker (FIG. 28).

This enrichment of CXCR4 in TOX3 MCF-7 stable transfectants is correlated with a gain-of-function result in migration studies. Whereas vector control cells displayed the same capacity for matrigel invasion in a transwell assay, with or without fetal calf serum, addition of serum to TOX3 MCF-7 stable transfectants resulted in a clear infiltration of cell aggregates (FIG. 29). These results further suggest that TOX3 role in migration is mediated, at least in part, by elevated CXCR4 expression in these cells.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the forms of TOX proteins, including TOX3 variants, method of detecting TOX proteins, sources of TOX protein or gene transcript expression, binding activities for TOX proteins and the techniques used to manufature or express TOX proteins, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### REFERENCES

1. Stratton M R, Rahman N. The emerging landscape of breast cancer susceptibility. Nat. Genet. 2008; 40(1):17-22.
2. Wilkinson B, Chen J Y, Han P, Rufner K M, Goularte O D, Kaye J. TOX: an HMG box protein implicated in the regulation of thymocyte selection. Nat. Immunol. 2002; 3(3):272-80.
3. van Gent D C, Hiom K, Paull T T, Gellert M. Stimulation of V(D)J cleavage by high mobility group proteins. Embo J. 1997; 16(10):2665-70.
4. Wang W, Chi T, Xue Y, Zhou S, Kuo A, Crabtree G R. Architectural DNA binding by a high-mobilitygroup/kinesin-like subunit in mammalian SWI/SNF-related complexes. Proc Natl Acad Sci USA. 1998; 95(2):492-8.
5. O'Flaherty E, Kaye J. TOX defines a conserved subfamily of HMG-box proteins. BMC Genomics. 2003; 4(1): 13.
6. Yuan S H, Qiu Z, Ghosh A. TOX3 regulates calcium-dependent transcription in neurons. Proc Natl Acad Sci USA. 2009; 106(8):2909-14. PMCID: 2650364.
7. Kajitani T, Mizutani T, Yamada K, Yazawa T, Sekiguchi T, Yoshino M, et al. Cloning and characterization of granulosa cell high-mobility group (HMG)-box protein-1, a novel HMG-box transcriptional regulator strongly expressed in rat ovarian granulosa cells. Endocrinology. 2004; 145 (5):2307-18.
8. Liang S, Zhao S, Mu X, Thomas T, Klein W H. Novel retinal genes discovered by mining the mouse embryonic RetinalExpress database. Molecular vision. 2004; 10:773-86.
9. Aliahmad P, Kaye J. Development of all CD4 T lineages requires nuclear factor TOX. J Exp Med. 2008; 205(1):245-56.
10. Smid M, Wang Y, Klijn J G, Sieuwerts A M, Zhang Y, Atkins D, et al. Genes associated with breast cancer metastatic to bone. J Clin Oncol. 2006; 24(15):2261-7.
11. Stacey S N, Manolescu A, Sulem P, Rafnar T, Gudmundsson J, Gudjonsson S A, et al. Common variants on chromosomes 2q35 and 16q12 confer susceptibility to estrogen receptor-positive breast cancer. Nat. Genet. 2007; 39(7):865-9.
12. Easton D F, Pooley K A, Dunning A M, Pharoah P D, Thompson D, Ballinger D G, et al. Genome-wide association study identifies novel breast cancer susceptibility loci. Nature. 2007; 447(7148):1087-93.
13. Garcia-Closas M, Chanock S. Genetic susceptibility loci for breast cancer by estrogen receptor status. Clin Cancer Res. 2008; 14(24):8000-9. PMCID: 2668137.
14. James J J, Evans A J, Pinder S E, Gutteridge E, Cheung K L, Chan S, et al. Bone metastases from breast carcinoma: histopathological-radiological correlations and prognostic features. British journal of cancer. 2003; 89(4):660-5.
15. Latif A, Hadfield K D, Roberts S A, Shenton A, Lalloo F, Black G C, et al. Breast cancer susceptibility variants alter risks in familial disease. J Med. Genet. 2009.
16. Li L, Zhou X, Huang Z, Liu Z, Song M, Guo Z. TNRC9/LOC643714 polymorphisms are not associated with breast cancer risk in Chinese women. Eur J Cancer Prey. 2009; 18(4):285-90.
17. Song H, Ramus S J, Kjaer S K, DiCioccio R A, Chenevix-Trench G, Pearce C L, et al. Association between invasive ovarian cancer susceptibility and 11 best candidate SNPs from breast cancer genome-wide association study. Hum Mol. Genet. 2009; 18(12):2297-304.
18. Nordgard S H, Johansen F E, Alnaes G I, Naume B, Borresen-Dale A L, Kristensen V N. Genes harbouring susceptibility SNPs are differentially expressed in the breast cancer subtypes. Breast Cancer Res. 2007; 9(6):113.
19. Huijts P E, Vreeswijk M P, Kroeze-Jansema K H, Jacobi C E, Seynaeve C, Krol-Warmerdam E M, et al. Clinical correlates of low-risk variants in FGFR2, TNRC9, MAP3K1, LSP1 and 8q24 in a Dutch cohort of incident breast cancer cases. Breast Cancer Res. 2007; 9(6):R78.
20. Riaz M, Elstrodt F, Hollestelle A, Dehghan A, Klijn J G, Schutte M. Low-risk susceptibility alleles in 40 human breast cancer cell lines. BMC Cancer. 2009; 9:236.
21. Antoniou A C, Spurdle A B, Sinilnikova O M, Healey S, Pooley K A, Schmutzler R K, et al. Common breast cancer-predisposition alleles are associated with breast cancer risk in BRCA1 and BRCA2 mutation carriers. Am J Hum Genet. 2008; 82(4):937-48.
22. Lakhani S R, Van De Vijver M J, Jacquemier J, Anderson T J, Osin P P, McGuffog L, et al. The pathology of familial breast cancer: predictive value of immunohistochemical markers estrogen receptor, progesterone receptor, HER-2, and p53 in patients with mutations in BRCA1 and BRCA2. J Clin Oncol. 2002; 20(9):2310-8.
23. Rae J M, Johnson M D, Scheys J O, Cordero K E, Larios J M, Lippman M E. GREB 1 is a critical regulator of hormone dependent breast cancer growth. Breast Cancer Res Treat. 2005; 92(2):141-9.
24. Neve R M, Chin K, Fridlyand J, Yeh J, Baehner F L, Fevr T, et al. A collection of breast cancer cell lines for the study of functionally distinct cancer subtypes. Cancer Cell. 2006; 10(6):515-27. PMCID: 2730521.
25. Chin K, DeVries S, Fridlyand J, Spellman P T, Roydasgupta R, Kuo W L, et al. Genomic and transcriptional aberrations linked to breast cancer pathophysiologies. Cancer Cell. 2006; 10(6):529-41.
26. Sorlie T, Perou C M, Tibshirani R, Aas T, Geisler S, Johnsen H, et al. Gene expression patterns of breast carcinomas distinguish tumor subclasses with clinical implications. Proc Natl Acad Sci USA. 2001; 98(19):10869-74. PMCID: 58566.
27. Schneider J, Ruschhaupt M, Buness A, Asslaber M, Regitnig P, Zatloukal K, et al. Identification and meta-analysis of a small gene expression signature for the diagnosis of estrogen receptor status in invasive ductal breast cancer. Int J. Cancer. 2006; 119(12):2974-9.
28. Nakshatri H, Srour E F, Badve S. Breast cancer stem cells and intrinsic subtypes: controversies rage on. Curr Stem Cell Res Ther. 2009; 4(1):50-60.
29. Bertucci F, Loriod B, Nasser V, Granjeaud S, Tagett R, Braud A C, et al. Gene expression profiling of breast carcinomas using nylon DNA arrays. C R Biol. 2003; 326(10-11):1031-9.
30. Chanrion M, Fontaine H, Rodriguez C, Negre V, Bibeau F, Theillet C, et al. A new molecular breast cancer subclass defined from a large scale real-time quantitative RT-PCR study. BMC Cancer. 2007; 7:39. PMCID: 1828062.
31. Wang Y, Rathinam R, Walch A, Alahari S K. ST14 (suppression of tumorigenicity 14) gene is a target for miR-27b, and the inhibitory effect of ST14 on cell growth is independent of miR-27b regulation. J Biol. Chem. 2009; 284(34):23094-106.
32. Jacquemier J, Ginestier C, Rougemont J, Bardou V J, Charafe-Jauffret E, Geneix J, et al. Protein expression profiling identifies subclasses of breast cancer and predicts prognosis. Cancer Res. 2005; 65(3):767-79.
33. Herynk M H, Lewis M T, Hopp T A, Medina D, Corona-Rodriguez A, Cui Y, et al. Accelerated mammary maturation and differentiation, and delayed MMTVneu-induced tumorigenesis of K303R mutant ERalpha transgenic mice. Oncogene. 2009; 28(36):3177-87.
34. Sinn E, Muller W, Pattengale P, Tepler I, Wallace R, Leder P. Coexpression of MMTV/v-Ha-ras and MMTV/c-myc genes in transgenic mice: synergistic action of oncogenes in vivo. Cell. 1987; 49(4):465-75.
35. Tsukamoto A S, Grosschedl R, Guzman R C, Parslow T, Varmus H E. Expression of the int-1 gene in transgenic mice is associated with mammary gland hyperplasia and adenocarcinomas in male and female mice. Cell. 1988; 55(4):619-25.
36. Richert M M, Schwertfeger K L, Ryder J W, Anderson S M. An atlas of mouse mammary gland development. J Mammary Gland Biol Neoplasia. 2000; 5(2):227-41.
37. Guy C T, Webster M A, Schaller M, Parsons T J, Cardiff R D, Muller W J. Expression of the neu-protooncogene in the mammary epithelium of transgenic mice induces metastatic disease. Proc Natl Acad Sci USA. 1992; 89(22):10578-82. PMCID: 50384.
38. Muller W J, Sinn E, Pattengale P K, Wallace R, Leder P. Single-step induction of mammary adenocarcinoma in transgenic mice bearing the activated c-neu oncogene. Cell. 1988; 54(1):105-15.
39. Antoniou A C et al. Common breast cancer-predisposition alleles are associated with breast cancer risk in BRCA1 and BRCA2 mutation carriers. Am J Hum Genet. 2008; 82(4):937-48.
40. Stevens K N et al. Common Breast Cancer Susceptibility Loci Are Associated with Triple-Negative Breast Cancer Cancer Res. 2011; 71 (19): 6240-6249.
41. Orr N, Cooke R, Jones M, Fletcher O, Dudbridge F, Chilcott-Burns S, Tomczyk K, Broderick P, Houlston R, Ashworth A, Swerdlow A.Genetic Variants at Chromosomes 2q35, 5p12, 6q25.1, 10q26.13, and 16q12.1. Influence the Risk of Breast Cancer in Men. PLoS Genet. 2011; 7(9) (ePub).
42. Sorlie T, Tibshirani R, Parker J, Hastie T, Marron J S, Nobel A, Deng S, Johnsen H, Pesich R, Geisler S, Demeter J, Perou C M, Lonning P E, Brown P O, Børresen-Dale A L, Botstein D. Repeated observation of breast tumor subtypes in independent gene expression data sets. Proc Natl Acad Sci USA. 2003; 100(14):8418-23.
43. Guedj M, Marisa L, de Reynies A, Orsetti B, Schiappa R, Bibeau F, Macgrogan G, Lerebours F, Finetti P, Longy M, Bertheau P, Bertrand F, Bonnet F, Martin A L, Feugeas J P, Bièche I, Lehmann-Che J, Lidereau R, Birnbaum D, Bertucci F, de Thé H, Theillet C. A refined molecular taxonomy of breast cancer. Oncogene. 2011 (ePub).
44. Lefebvre O, Chenard M P, Masson R, Linares J, Dierich A, LeMeur M, Wendling C, Tomasetto C, Chambon P, Rio M C. Gastric mucosa abnormalities and tumorigenesis in mice lacking the pS2 trefoil protein. Science. 1996; 274(5285):259-62.
45. Amiry N, Kong X, Muniraj N, Kannan N, Grandison P M, Lin J, Yang Y, Vouyovitch C M, Borges S, Perry J K, Mertani H C, Zhu T, Liu D, Lobie P E. Trefoil factor-1 (TFF1) enhances oncogenicity of mammary carcinoma cells. Endocrinology. 2009; 150(10):4473-83.
46. Welboren W J, van Driel M A, Janssen-Megens E M, van Heeringen S J, Sweep F C, Span P N, Stunnenberg H G. ChIP-Seq of ERalpha and RNA polymerase II defines genes differentially responding to ligands. The EMBO Journal. 2009; 28:1418-1428.
47. Buache E, Etique N, Alpy F, Stoll I, Muckensturm M, Reina-San-Martin B, Chenard M P, Tomasetto C, Rio M C. Deficiency in trefoil factor 1 (TFF1) increases tumorigenicity of human breast cancer cells and mammary tumor development in TFF 1-knockout mice. Oncogene. 2011; 30(29):3261-73.

1. (canceled)

2. (canceled)

3. (canceled)

4. (canceled)

5. (canceled)

6. (canceled)

7. (canceled)

8. A method of determining the subtype of cancer in a subject, comprising:
obtaining a test sample from a subject;
determining the expression level of at least one biomarker in the test sample;
comparing the expression level of the at least one biomarker in the test sample with the expression level the at least one biomarker in a reference sample from a healthy individual; and
determining that the subject has a particular subtype of cancer based on the level of expression of the at least one biomarker in the test sample compared to the level of expression of the at least one biomarker in the reference sample from the healthy individual.

9. The method of claim 8, wherein the test sample comprises a tissue or a cell.

10. The method of claim 8, wherein the at least one biomarker is TOX3, TFF1, TFF3, AGR2, SCUBE2, CEACAM6, TSPAN1 and/or CXCR4.

11. The method of claim 8, wherein determining the expression level of the at least one biomarker comprises analyzing the transcription level of the at least one biomarker or analyzing the protein level of the at least one biomarker.

12. The method of claim 8, wherein the cancer is breast cancer.

13. The method of claim 8, wherein the cancer is a subset of breast cancer.

14. The method of claim 13, wherein the subset of breast cancer is luminal A, luminal B, luminal C, molecular-apocrine, basal, or normal-breast-like cancer.

15. The method of claim 13, wherein the subset of breast cancer is tamoxifen resistant.

16. The method of claim 13, wherein the subset of breast cancer is fulvestant sensitive.

17. A method of determining an increased susceptibility of a subject to cancer, comprising:
obtaining a test sample from the subject;
determining the expression level of at least one biomarker in the test sample;
comparing the expression level of the at least one biomarker in the test sample with the expression level of the at least one biomarker in a reference sample from a healthy individual; and
determining that the subject has an increased susceptibility to cancer based on the level of expression of the at least one biomarker in the test sample compared to the level of expression of the at least one biomarker in the reference sample from the healthy individual.

18. The method of claim 17, wherein the sample comprises a tissue or a cell.

19. The method of claim 17, wherein the at least one biomarker is TOX3, TFF1, TFF3, AGR2, SCUBE2, CEACAM6, TSPAN1 and/or CXCR4.

20. The method of claim 17, wherein determining the expression level of the at least one biomarker comprises analyzing the transcription level of the at least one biomarker or analyzing the protein level of the at least one biomarker.

21. The method of claim 17, wherein the cancer is breast cancer.

22. The method of claim 17, wherein the cancer is a subset of breast cancer.

23. The method of claim 22, wherein the subset of breast cancer is luminal A, luminal B, luminal C, molecular-apocrine, basal, or normal-breast-like cancer.

24. The method of claim 22, wherein the subset of breast cancer is tamoxifen resistant.

25. The method of claim 22, wherein the subset of breast cancer is fulvestant sensitive.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method of selecting a treatment for a cancer patient, comprising:
assaying a biological sample from the patient by
detecting the expression level of at least one biomarker in the test sample;
comparing the expression level of the at least one biomarker in the test sample with the expression level the at least one biomarker in a reference sample from a healthy individual; and
determining that the subject has a particular subtype of cancer based on the level of expression of the at least one biomarker in the test sample compared to the level of expression of the at least one biomarker in the reference sample from the healthy individual; and
based on that determination, selecting a treatment for the patient.

41. The method of claim 40, wherein the at least one biomarker is TOX3, TFF1, TFF3, AGR2, SCUBE2, CEACAM6, TSPAN1 and/or CXCR4.

42. The method of claim 40, wherein determining the expression level of the at least one biomarker comprises analyzing the transcription level of the at least one biomarker or analyzing the protein level of the at least one biomarker.

43. The method of claim 40, wherein the cancer is breast cancer.

44. The method of claim 40, wherein the cancer is a subset of breast cancer.

45. The method of claim 44, wherein the subset of breast cancer is luminal A, luminal B, luminal C, molecular-apocrine, basal, or normal-breast-like cancer.

46. The method of claim 40, wherein selecting a treatment includes selection of tamoxifen and/or fulvestant.


SEQUENCE LISTING (6 sequences)

SEQ ID NO: 1 - protein, 589 aa, Homo sapiens
MDVRFYPAAAGDPASLDFAQCLGYYGYSKFGNNNNYMNMAEANNAFFAASEQTFHTPSLGDEEFEIPPITPPPESDPALGMPDVLLPFQALSDPLPSQGSEFTPQFPPQSLDLPSITISRNLVEQDGVLHSSGLHMDQSHTQVSQYRQDPSLIMRSIVHMTDAARSGVMPPAQLTTINQSQLSAQLGLNLGGASMPHTSPSPPASKSATPSPSSSINEEDADEANRAIGEKRAAPDSGKKPKTPKKKKKKDPNEPQKPVSAYALFFRDTQAAIKGQNPNATFGEVSKIVASMWDSLGEEQKQVYKRKTEAAKKEYLKALAAYRASLVSKAAAESAEAQTIRSVQQTLASTNLTSSLLLNTPLSQHGTVSASPQTLQQSLPRSIAPKPLTMRLPMNQIVTSVTIAANMPSNIGAPLISSMGTTMVGSAPSTQVSPSVQTQQHQMQLQQQQQQQQQQMQQMQQQQLQQHQMHQQIQQQMQQQHFQHHMQQHLQQQQQHLQQQINQQQLQQQLQQRLQLQQLQHMQHQSQPSPRQHSPVASQITSPIPAIGSPQPASQQHQSQIQSQTQTQVLSQAIPTICESNCLMNPGTY

SEQ ID NO: 2 - protein, 238 aa, Homo sapiens
MDVRFYPAAAGDPASLDFAQCLGYYGYSKFGNNNNYMNMAEANNAFFAASEQTFHTPSLGDEEFEIPPITPPPESDPALGMPDVLLPFQALSDPLPSQGSEFTPQFPPQSLDLPSITISRNLVEQDGVLHSSGLHMDQSHTQVSQYRQDPSLIMRSIVHMTDAARSGVMPPAQLTTINQSQLSAQLGLNLGGASMPHTSPSPPASKSATPSPSSSINEEDADEANRAIGEKRAAPDSG

SEQ ID NO: 3 - DNA, 1734 bp, Rattus norvegicus
atggatgtgaggttctaccccgcggcggccggggatcccgccggcctggacttcgcgcagtgcctggggtactacggctacagcaagttgggaaataataactacatgaacatggctgaggcaaacaacgccttctttgctgccagtgagcagacattccacacgccaagccttggggatgaagagtttgaaattccgccgatcacgcctcctccagagtcagaccccaccctgggcatgcccgatgtactgctaccctttcagacactcagcgatccgttgccttcccagggaaatgagttcacaccccagtttccccctcagagcctggatcttccttccatcacaatctcaaggaatctggtggagcaagatggtgtgcttcatagcaacgggctgcatatggatcagagccacacacaagtgtcgcagtaccgccaggatccttctttggtcatgaggtcaattgtccacatgacagatgctgctcgctctgggatcatgcctcctgcccaactgaccaccatcaaccagtctcagctcagtgcacagttgggcttgaatctgggaggagccagtgtgccccacacgtctccttcacctccagcaagcaaatcagccactccctccccttccagctctatcaatgaagaggatgctgatgaaacaaacagagccgttggagagaaaagaacagccccagattctggcaagaagcccaagactccaaagaaaaagaaaaagaaagatcccaacgagcctcagaagccagtgtcagcatacgccctgtttttcagagatacacaggctgcaattaaaggtcaaaaccccaatgcgacctttggagaagtctcaaaaattgtagcatctatgtgggacagccttggagaggagcaaaagcaggtatataaaaggaaaacagaagctgccaagaaagaatatttgaaggccctggctgcctaccgggccagcctcgtttctaaggctgctgctgagtccgcagaagcccagactatccgctctgtccagcagactctggcatcaaccaacctgacatcctctctccttctgaacacgtcactgtctcaacatgggacagtcccggcttcacctcagactctcccgcagtcactccctaggtcgattgcccccaaacccttaaccatgagactacccatgagccagattgtcacatcagtcaccattgcagccaacatgccctcgaacattggggctccacttatcagctccatgggaacgaccatggttggctcagtatcctccacgcaggtgagcccttcggtacaaacccagcaacatcagctgcagctgcagcagcagcaacaacagcagcagcagcagatgcagcagatgcaacatcagcagctgcagcagcaccagatgcatcagcagattcagcagcagatgcagcagcagcatttccagcaccacatgcaacagcacctgcagcagcagcaacagcagcacctccagcagcagatcagccaacagcagctgcagcagcagctgcagcagcatctccagctgcagcagcagctgcagcacatgcagcaccagtctcagccttctccccggcagcactcgcccgtcacctcacagatcacgtcccccatccccgccattggcagcccccagccagcctctcagcagcaccagcctcaaatccagtcgcagacacagactcaagtgttaccgcaggtcagtattttttaa

SEQ ID NO: 4 - DNA, 2849 bp, Homo sapiens
gcggccgcggctcccgagctcctcgggctctgggtcccggcgcccctccggccgcgagtcccacgcgccacccccgggcgccctcgacggtggatctagcggcggcgaggaggcgggtcccggccccggcgaaccccagtcccggcccccggccccgggcccagcttcggcatggatgtgaggttctaccccgcggcggccggggaccctgccagcctggacttcgcgcagtgcctggggtactacggctacagcaagtttggaaataataataactatatgaatatggctgaggcgaacaatgcgttcttcgctgccagtgagcagacattccacacaccaagccttggggacgaggaattcgaaattccaccaatcacgcctcctccagagtcagaccctgccctaggcatgccggatgtactgctaccctttcaagccctcagcgatccattgccttcccagggaagtgaattcacaccccagtttccccctcaaagcctggacctcccttccattacaatctcaagaaatctcgtggaacaagatggcgtgcttcatagcagtgggttgcatatggatcagagccacacacaagtgtcccagtaccggcaggatccctccctgatcatgcggtccatcgtccacatgaccgatgctgcgcgttctggggtcatgcctcctgcccagctcaccaccatcaaccagtctcagctcagcgcccagttggggttgaatttgggaggtgccagtatgcctcacacatctccttcacctccagcaagcaaatcagccactccctccccttccagctccatcaatgaagaggatgctgatgaagccaacagagccattggagagaaaagagctgctccagactctggcaagaagcccaagactccaaagaaaaagaaaaagaaagatcccaatgagccacagaagccagtgtcagcatatgccctgtttttcagagacacacaggctgcaattaaaggtcaaaaccccaatgcaacctttggagaggtctcaaaaattgtagcatctatgtgggacagccttggagaagaacaaaagcaggtatataaaaggaaaacagaagctgccaaaaaagaatacctgaaggccctggcggcatacagggccagcctcgtttctaaggctgctgctgagtcagcagaagcccagaccatccgttctgttcagcagaccctggcgtcgaccaatctaacatcctctctccttctcaacactccactgtctcaacatggaacagtgtcagcatcacctcagactctccagcaatccctccctaggtcaatcgctcccaaacccttaaccatgagactccccatgaaccagattgtcacatcagtcaccattgcagccaacatgccctcgaacattggggctccactgataagctccatgggaacgaccatggttggctcagcaccctccacccaagtgagtccttcggtgcaaacccagcagcatcagatgcaattgcagcagcagcagcagcagcaacaacaacagatgcaacagatgcagcagcagcaactccagcagcaccaaatgcatcagcaaatccagcagcagatgcagcagcagcatttccagcaccacatgcagcagcacctgcagcagcagcagcagcatctccagcagcaaattaatcaacagcagctgcagcagcagctgcagcagcgcctccagctgcagcagctgcaacacatgcagcaccagtctcagccttctcctcggcagcactcccctgtcgcctctcagataacatcccccatccctgccatcgggagcccccagccagcctctcagcagcaccagtcgcaaatacagtctcagacacagactcaagtattatcgcaggctatacctacaatatgtgaatcaaactgtttaatgaatcctgggacatactgatgactataaactggcctctctgagtcatagaaaaatggccttatttctccagaagtgagtaaaccacacttccaggctatctgaactcctgaagccctaaaaataaaaagcacagttgtaactacctgaaatatgaagatccagtttcatacaaacatttgtatgacgtgaatagttgatggcatttttttgtcatgaaaaaaataatgtaaatcacagacttttgccaaagctcttattttttttcctaaatctctccagaaaaaaaatgcaagtgactaaattcaattattgactaatttccactttttatccatgacttctccaaatcaaaccacagtatatgttgtaacaatatctatgaccactgttagcccattatattcattccaattagaagaaatgtgaatactatattccgtgttttgagtgacaagtttcgaaaaataaaaacactgtatttttaaaagggaaatgcacttaaatgaaaacagttattacaaaagttaagatttaaaaagaaaaagcaagagtttttattatgatgtaataccagtagaatatttaaaaggcacaccacatctgaataatcaatgtaaatattttctttcaaagttgtaagttttcatatcatgtgctgtaaagttttcctaaatgaggctttaacgtaaacactggtgacataaaccattcattgctacgttgcttattgtgtttttatgctgttttatacttttttatgagttatgatagcagcaattaagttgtttgtattttgcttaactaaaacaaaaatgcttttatcttgctatagaataaacacatttcagtaaaaactgtggactgtattttgatgcaacaacaaagaaactgttcacttttcaaataaaatgatatgtcagatttca

SEQ ID NO: 5 - protein, 571 aa, Homo sapiens
MKCQPRSGARRIEERLHYLITTYLKFGNNNNYMNMAEANNAFFAASETFHTPSLGDEEFEIPPITPPPESDPALGMPDVLLPFQALSDPLPSQGSEFTPQFPPQSLDLPSITISRNLVEQDGVLHSSGLHMDQSHTQVSQYRQDPSLIMRSIVHMTDAARSGVMPPAQLTTINQSQLSAQLGLNLGGASMPHTSPSPPASKSATPSPSSSINEEDADEANRAIGEKRAAPDSGKKPKTPKKKKKKDPNEPQKPVSAYALFFRDTQAAIKGQNPNATFGEVSKIVASMWDSLGEEQKQVYKRKTEAAKKEYLKALAAYRASLVSKAAAESAEAQTIRSVQQTLASTNLTSSLLLNTPLSQHGTVSASPQTLQQSLPRSIAPKPLTMRLPMNQIVTSVTIAANMPSNIGAPLISSMGTTMVGSAPSTQVSPSVQTQQHQMQLQQQQQQQQQQMQQMQQQQLQQHQMHQQIQQQMQQQHFQHHMQQHLQQQQQHLQQQINQQQLQQQLQQRLQLQQLQHMQHQSQPSPRQHSPVASQITSPIPAIGSPQPASQQHQSQIQSQTQTQVLSQVSIF

SEQ ID NO: 6 - DNA, 3123 bp, Homo sapiens
gaaccgacacgaggcttcacctgggaagcttcaagtctgcctacctgtgaaaggtcaggccccaacaccccttctgggaaatcctacagctaggatgcatttctctcactgaaccccatccagcagaggacagaagagtcagaagagggtagagaggatttagatactcatagaagatgtagtggaggatgaagtgccaacctcgctcgggagccaggcgcattgaggagagacttcattacctgataactacctatctgaaatttggaaataataataactatatgaatatggctgaggcgaacaatgcgttcttcgctgccagtgagacattccacacaccaagccttggggacgaggaattcgaaattccaccaatcacgcctcctccagagtcagaccctgccctaggcatgccggatgtactgctaccctttcaagccctcagcgatccattgccttcccagggaagtgaattcacaccccagtttccccctcaaagcctggacctcccttccattacaatctcaagaaatctcgtggaacaagatggcgtgcttcatagcagtgggttgcatatggatcagagccacacacaagtgtcccagtaccggcaggatccctccctgatcatgcggtccatcgtccacatgaccgatgctgcgcgttctggggtcatgcctcctgcccagctcaccaccatcaaccagtctcagctcagcgcccagttggggttgaatttgggaggtgccagtatgcctcacacatctccttcacctccagcaagcaaatcagccactccctccccttccagctccatcaatgaagaggatgctgatgaagccaacagagccattggagagaaaagagctgctccagactctggcaagaagcccaagactccaaagaaaaagaaaaagaaagatcccaatgagccacagaagccagtgtcagcatatgccctgtttttcagagacacacaggctgcaattaaaggtcaaaaccccaatgcaacctttggagaggtctcaaaaattgtagcatctatgtgggacagccttggagaagaacaaaagcaggtatataaaaggaaaacagaagctgccaaaaaagaatacctgaaggccctggcggcatacagggccagcctcgtttctaaggctgctgctgagtcagcagaagcccagaccatccgttctgttcagcagaccctggcgtcgaccaatctaacatcctctctccttctcaacactccactgtctcaacatggaacagtgtcagcatcacctcagactctccagcaatccctccctaggtcaatcgctcccaaacccttaaccatgagactccccatgaaccagattgtcacatcagtcaccattgcagccaacatgccctcgaacattggggctccactgataagctccatgggaacgaccatggttggctcagcaccctccacccaagtgagtccttcggtgcaaacccagcagcatcagatgcaattgcagcagcagcagcagcagcaacaacaacagatgcaacagatgcagcagcagcaactccagcagcaccaaatgcatcagcaaatccagcagcagatgcagcagcagcatttccagcaccacatgcagcagcacctgcagcagcagcagcagcatctccagcagcaaattaatcaacagcagctgcagcagcagctgcagcagcgcctccagctgcagcagctgcaacacatgcagcaccagtctcagccttctcctcggcagcactcccctgtcgcctctcagataacatcccccatccctgccatcgggagcccccagccagcctctcagcagcaccagtcgcaaatacagtctcagacacagactcaagtattatcgcaggtcagtattttctgaagacgcatatggcagacggatttgcgtataccaaggagagtggcataggagggaaaagcatatgtggctgaaacctgtaagttggtgttggttatgcagaaatgtgtaacagatcaaacggtcctctcaagtgtctattagataggcaataagaactgcagtgtagctgagtaacatcttttagctgactataaatcactttgtttttaaacaagaaaagctgtgctcttttatgtgatgccttttttatttattcaggctatacctacaatatgtgaatcaaactgtttaatgaatcctgggacatactgatgactataaactggcctctctgagtcatagaaaaatggccttatttctccagaagtgagtaaaccacacttccaggctatctgaactcctgaagccctaaaaataaaaagcacagttgtaactacctgaaatatgaagatccagtttcatacaaacatttgtatgacgtgaatagttgatggcatttttttgtcatgaaaaaaataatgtaaatcacagacttttgccaaagctcttattttttttcctaaatctctccagaaaaaaaatgcaagtgactaaattcaattattgactaatttccactttttatccatgacttctccaaatcaaaccacagtatatgttgtaacaatatctatgaccactgttagcccattatattcattccaattagaagaaatgtgaatactatattccgtgttttgagtgacaagtttcgaaaaataaaaacactgtatttttaaaagggaaatgcacttaaatgaaaacagttattacaaaagttaagatttaaaaagaaaaagcaagagtttttattatgatgtaataccagtagaatatttaaaaggcacaccacatctgaataatcaatgtaaatattttctttcaaagttgtaagttttcatatcatgtgctgtaaagttttcctaaatgaggctttaacgtaaacactggtgacataaaccattcattgctacgttgcttattgtgtttttatgctgttttatacttttttatgagttatgatagcagcaattaagttgtttgtattttgcttaactaaaacaaaaatgcttttatcttgctatagaataaacacatttcagtaaaaactgtggactgtattttgatgcaacaacaaagaaactgttcacttttcaaataaaatgatatgtcagatttc